(19) United States
(12) Patent Application Publication
(10) Pub. No.: US 2025/0041664 A1
(43) Pub. Date: Feb. 6, 2025

(54) System for using athletic activity as software input via a motion sensor with adaptability to multiple sensor positions

(71) Applicant: Paul Allen Tillery, IV, Raleigh, NC, NC (US)

(72) Inventor: Paul Allen Tillery, IV, Raleigh, NC, NC (US)

(21) Appl. No.: 18/793,749

(22) Filed: Aug. 2, 2024

Related U.S. Application Data
(60) Provisional application No. 63/517,547, filed on Aug. 3, 2023.

(51) Int. Cl. A63B 24/00; G01P 15/18; H04W 4/80

(52) U.S. Cl. CPC A63B 24/0062; G01P 15/18; A63B 2024/0071; H04W 4/80

(57) ABSTRACT

A method is provided for using any repetitive athletic activity as software input via a motion sensor assembly removably affixed to any limb segment moved by the activity in repeating swings. Embodiments may generate statistics about the intended activity, such as repetition count and cadence (e.g. cycling RPM), for use in triggering software response. Further embodiments may dynamically calibrate for various sensor assembly positions and exercise types (e.g. on the thigh or ankle for leg-pedaling, or on the wrist or upper arm for arm-pedaling, all at any roll about the limb segment's proximal-to-distal axial dimension). Preferred embodiments may handle calibration covertly as the user operates the software's intended purpose (without an overt “calibration phase,” and without needing to request information from the user). Further embodiments may track an intended activity (e.g. leg-pedaling) while identifying other motions (e.g. knee-swaying, knee-shaking, or sensor assembly disruptions) as distinct from the intended activity.

### CROSS-REFERENCE TO RELATED APPLICATIONS

This application claims the benefit of the filing date of my U.S. Provisional Patent Application Ser. No. 63/517,547, filed Aug. 3, 2023, the entire contents of which are incorporated herein by reference.

### DESCRIPTION

### Field

The invention relates to the fields of athletics and interactive software, and in particular to a method for using athletic activity statistics for software input via motion sensor data.

### Background

Prior methods for using athletic activity as software input are varied, but they pose disadvantages in terms of accessibility, flexibility, and user experience.

Accelerometer-based step counters are ubiquitous in mobile devices and useful for estimating steps taken in a session of athletic activity. However, their utility is limited for real-time software input. (They tend to be better suited for general estimates than precise tracking, and they may lack adaptability to various forms of low-impact activity.)

Standalone athletic equipment with built-in software (e.g. an exercise bike with an attached screen and pre-installed interactive programs) may offer more real-time precision, but may also be expensive, relatively inaccessible for developers (e.g. as compared to gaming consoles, personal computers, and mobile devices), and limited in program options for users (e.g. how many programs are available, and/or whether a single program may be usable across multiple pieces of equipment).

“Smart” athletic equipment capable of pairing with other devices (e.g. through Bluetooth or ANT+) may offer more flexibility, but may still pose limitations in terms of expense, developer accessibility, and device/platform compatibility.

Cadence meter peripherals may offer significantly more flexibility at lower cost; however, they may still lack platform compatibility, and if a user changes workout equipment (e.g. on separate trips to a gym), the process of attaching and removing a cadence meter for each activity session may be inconvenient.

Some fitness video games use motion sensor assemblies (e.g. mobile devices or game controllers with gyroscope and/or accelerometer components) worn or held by the user. This approach tends to offer a favorable balance of cost and convenience (for both users and developers), but its implementation thus far has still posed significant limitations.

For example, Nintendo™'s Ring Fit Adventure™ allows the user to move an avatar by running in place with a motion sensor assembly strapped to the left thigh. However, the resulting effect is generally limited to three tiers (stopped, slow, or fast), and it only works with the sensor assembly in one specific position (the sensor assembly must be front-and-center on the left thigh only, with other positions not viable for various software features and mechanics). Furthermore, Ring Fit Adventure™ frequently delays or interrupts gameplay in order for the user to confirm sensor assembly position or perform practice motions, creating a sense of separation between calibration phases and the software's main operation.

Collectively, prior methods of using athletic activity for software input pose various problems for both developers and users. For developers, prior methods may be inconvenient to develop for, incompatible with target platforms, and/or limited in precision. For users, prior methods may be expensive to access, difficult to use across varied activities or equipment types, and/or limited in adaptability to personal comfort or anatomical considerations.

### Summary

Embodiments of the present invention provide a method of using any repetitive athletic activity as input to a software routine running on a processor. Embodiments may use a motion sensor assembly interfaced with the processor, and the sensor assembly's hardware may include a gyroscope component and/or an accelerometer component. (Preferred embodiments may include both a gyroscope and an accelerometer in the sensor assembly, though alternative embodiments may operate with just one or the other.) Each component may provide readings with values on three local Cartesian axes, and the sensor assembly can be affixed on any limb segment moved by the activity in repeating swings. Embodiments may be adaptable to any sensor assembly attachment position and orientation relative to the limb segment, though the software routine's developer may design some embodiments specifically within developer intentions for attachment guidelines or limitations. Embodiments may calculate a variety of statistics about the activity (including specific numeric values for cadence and total repetition count), any or all of which may be used as input for the software routine.

These embodiments solve the aforementioned problems through compatibility with off-the-shelf motion-sensing devices (e.g. mobile phones and video game controllers featuring gyroscope components, and optionally accelerometer components as well), adaptability to multiple sensor assembly positions (e.g. at any orientation on the thigh or ankle for activity involving leg motion), viability with a variety of activity types and equipment (i.e. without need for equipment-specific software, “smart” capabilities, etc.), and instant operability without an overt calibration phase (i.e. with calibration preferably handled discreetly as the user begins operating the software, not requiring the user to go through an announced calibration phase before the software's main operation).

Embodiments may gather calibration data by at least one of three calibration methods, any of which may preferably execute without needing to be announced to the user.

A first calibration method may designate the sensor assembly's axes as proximal, perpendicular, and transverse, and may ascertain aspects of the sensor assembly's orientation relative to the limb segment. In this calibration method, the proximal, perpendicular, and transverse axes may further be identified as primary, secondary, and/or tertiary (depending on the extents to which they are expected to detect motion during the intended activity), and the axes may also have sign multipliers determined (with “sign multipliers” referring to signifiers of whether values on an axis should be multiplied by −1 when determining direction of motion). The workings of the first calibration method are described in greater detail below.

A second calibration method (operable with the sensor assembly at any orientation relative to the limb segment) may sample motion data on multiple axes to determine expected relationships between measurements on the axes during performance of the intended activity. This may determine primary, secondary, and/or tertiary axis identifications, and may also determine primary-secondary, primary-tertiary, and/or secondary-tertiary sign multipliers. The workings of the second calibration method are described in greater detail below.

A third calibration method (also operable with the sensor assembly at any orientation relative to the limb segment) may determine, on an axis-by-axis basis, whether to monitor measurements on an axis when tracking the intended activity. The workings of the third calibration method are described in greater detail below.

Given calibration results, embodiments may detect progressions from one swing to another by detecting changes in direction of the sensor assembly's motion (with each direction change indicating the completion of one swing and the start of another), or by detecting the mid-point of each swing (with each mid-point detection indicating the activity has progressed from one swing to another since the previous mid-point detection). Mid-point detection operates on the assumption that motion on an axis measuring the swing is slowest when the sensor assembly is changing directions between swings, and fastest in the middle of each swing; therefore a midpoint may be detected as the point when an axis has peaked in speed during the swing (with the peak indicated by a high rate of gyroscopic rotation and/or a low change in linear acceleration).

For detecting swing transitions, embodiments may detect direction-changes using an interpreted gyroscopic value, which may be calculated each reading. The interpreted gyroscopic value may be derived from multiple axes by either a first gyroscopic interpretation method (based on sensor assembly orientation) or a second gyroscopic interpretation method (based on a hierarchy of the axes). The interpreted gyroscopic value's absolute value may indicate the reading's degrees rotated, and the interpreted gyroscopic value's positive or negative sign may indicate direction rotated. Therefore direction-changes may be detected when interpreted gyroscopic values change in sign from positive to negative (or vice versa). The workings of the two gyroscopic interpretation methods are described in greater detail below.

Further embodiments may detect swing transitions by detecting when gyroscopic values on any individual axis change in sign from positive to negative (or vice versa), or detecting when gyroscopic values on any axis transition from decreasing in absolute value to increasing in absolute value from one reading to another (with these absolute values decreasing as rotation slows while changing direction at the end of one swing, and increasing as rotation speeds up in the other direction at the start of a new swing).

Further embodiments may instead use accelerometer readings to detect the directional changes of each swing transition. This may be detected when accelerometer value deltas between one reading and another on an axis change in sign from positive to negative (with positive accelerometer deltas indicating the process of changing direction, and negative accelerometer deltas indicating continuation in a new direction), or when accelerometer values on an axis transition from getting further from their ongoing average value to getting closer to their ongoing average value (with accelerometer values expected to be furthest from their ongoing average when changing direction).

For detecting swing mid-points, embodiments may detect the peak speed during a swing when gyroscopic values on any axis transition from increasing in absolute value to decreasing in absolute value from one reading to another. Alternative embodiments may instead detect mid-points when accelerometer value deltas between one reading and another on an axis transition from decreasing in absolute value to increasing in absolute value (with negative accelerometer deltas indicating continuation in a direction, and with positive accelerometer deltas indicating the process of changing direction), and (c) detecting when accelerometer values on an axis transition from getting closer to their ongoing average value to getting further from their ongoing average value (with accelerometer values expected to be closest to their ongoing average at the mid-point between direction changes).

Using the data and swing detections described above, embodiments may calculate a variety of statistics about the athletic activity. Repetition count may be calculated as total completed swings divided by the number of swings per repetition. A relative angle of the sensor assembly may also be calculated, starting at a reference value when the software initiates (which may be set by developer preference, or based on orientation data from the first calibration method), and then be calculated for each reading as the prior relative angle plus degrees rotated the reading. Each swing's range-of-motion may be calculated as either the sum of degrees rotated in the readings during the swing, or the difference between the highest and lowest relative angles of the sensor assembly during a swing. Each swing's swing rotation rate may be calculated as the swing's range-of-motion divided by its duration. Recent swing ranges may be cached as the range-of-motion values of at least the last two completed swings. An expected range-of-motion may be calculated as an average of recent swing ranges. Half-swing durations may be calculated as the time taken for a swing's mid-point to be detected, or the time taken for a swing's range-of-motion value to exceed half the expected range-of-motion value. Swing durations may be calculated as time elapsed between a swing's start and completion, or as time elapsed between two consecutive detections of swing mid-points. Repetition durations may be calculated by either determining time elapsed while performing the number of swings per repetition, calculating a single swing's duration multiplied by the number of swings per repetition, or calculating twice a half-swing's duration multiplied by the number of swings per repetition. Cadence may be calculated as either a unit of time numerator divided by repetition duration, a unit of time numerator divided by the number of swings per repetition then further divided by swing duration, or half a unit of time numerator divided by the number of swings per repetition then further divided by half-swing duration.

Embodiments may output any or all of these statistics input to the software routine. Example embodiments may use this input for conveying the calculated statistics numerically, displaying the calculated statistics graphically, adjusting a digital avatar's posture, adjusting a digital avatar's motion in virtual space, triggering interactions with virtual characters, props, and/or environments, triggering interactions with user interface elements, and/or triggering interactive audio-visual playback responses.

In the first calibration method, embodiments may designate the sensor assembly's axes as proximal, transverse, and perpendicular. (The proximal axis runs primarily along the limb segment's proximal-to-distal axial dimension, the perpendicular axis passes perpendicularly through the limb segment, and the transverse axis is orthogonal to the other two with its axial line tangential to the limb segment's surface.) The first calibration method may ascertain aspects of sensor assembly orientation; these aspects may include local positive directions of gyroscopic rotation and/or linear acceleration for each axis, the sensor assembly's pitch, and the sensor assembly's roll about the proximal axis.

Embodiments may use these orientation aspects to facilitate a variety of determinations, including primary, secondary, and/or tertiary axis identifications for the proximal, perpendicular, and/or transverse axes (with the primary axis expected to detect the most motion during performance of the intended activity, and the tertiary axis expected to detect the least). The proximal, perpendicular, and/or transverse axes may also each be given a sign multiplier that signifies, based on the orientation relative to the user of local positive rotation on the axis, whether gyroscopic rotation on the axis should be multiplied by −1 for a positive result to indicate rotation in a known direction relative to the user and a negative result to indicate rotation in the opposite direction relative to the user.

The orientation aspects may be ascertained by any combination of developer-intended instructions, estimations via readings from the sensor assembly (as described below), and/or indicators reported by the user. (It should be noted that requesting orientation indicators from the user, while potentially convenient for the developer, may be seen by the user as a hinderance, so preferred embodiments may operate without needing any user-reported orientation indicators.)

While varying embodiments may take different approaches to sensor assembly orientation, preferred embodiments may use developer-intended instructions for how to orient the sensor assembly's local positive directions relative to the limb segment (e.g. instructing the user to make sure a certain feature on the sensor assembly is pointed in the limb segment's proximal direction, and/or that another feature on the sensor assembly is pointed perpendicularly outward from the limb segment). These embodiments may still give the user freedom to place the sensor assembly on a limb segment with any pitch (e.g. on a horizontal thigh or an upright shin) and at any roll about the proximal axis (e.g. facing front, back, left, or right from the user's perspective, or any diagonal mix thereof). Preferred embodiments may then use readings from the sensor assembly to estimate the pitch and roll, all without needing to request information from the user.

Embodiments may estimate the extent to which the sensor assembly's pitch is upright via accelerometer response to the pull of gravity on the sensor assembly's axes, with stronger gravitational pull on the proximal axis indicating a pitch that is more upright, and stronger gravitational pull on the non-proximal axes indicating a pitch that is less upright. To isolate response to the pull of gravity as opposed to linear acceleration from user motion, embodiments may use averages of accelerometer readings over time on each axis. Embodiments may express the pitch estimation as a numeric value indicating how upright the pitch is, and/or as a toggleable value indicating whether the pitch is upright or angled (with “angled” referring to any pitch that is not considered upright) as determined using an upright/angled threshold. (The specific threshold between upright and angled may be set by the developer, and may be adjustable by the user via software settings if the developer allows.)

If the local positive directions of linear acceleration are known for the perpendicular and transverse axes, embodiments may estimate roll via readings from the sensor assembly, though the means of this estimation may vary depending on the sensor assembly's pitch.

If the sensor assembly's pitch is sufficiently angled for gravity to pull in a consistent direction on the perpendicular axis, the transverse axis, or both, then the sensor assembly's roll about the proximal axis may be estimated based on gravity's pull on the perpendicular and transverse axes relative to their local positive directions of linear acceleration. (For example, if the pull of gravity is primarily detected on the transverse axis, that indicates the sensor assembly's roll is oriented to one side or the other. If accelerometer readings on this axis are positive, and the local positive direction of linear acceleration for the axis is known to be clockwise about the limb segment from the user's perspective, that indicates the roll is left-facing.)

If the sensor assembly's pitch is upright to the point that gravity does not pull in a consistent direction on the perpendicular or transverse axis, and the intended activity moves the sensor assembly in forward swings with a high mid-point and backward swings with a low mid-point (e.g. on a user's ankle during a pedaling motion), then the sensor assembly's roll about the proximal axis may be estimated through analysis of linear acceleration and gyroscopic rotation. This approach may identify swing directions as forward or backward by checking which of the last two completed swings more strongly indicated a downward-to-upward direction change (with such a change being signified by acceleration on the proximal axis against the pull of gravity, and with the swing that more strongly indicated a downward-to-upward direction change being identified as backward and vice versa). This approach may then use three evaluations for whether the sensor assembly's roll is orthogonal-or-diagonal, front-or-back, and left-or-right. The orthogonal-or-diagonal evaluation (with “orthogonal” referring to regions of orientation the developer has deemed primarily front, back, left, or right, and “diagonal” referring to regions of orientation the developer has deemed mixes of front-or-back and left-or-right), may check the extent to which rotation on one non-proximal axis exceeds rotation on the other non-proximal axis, with a result of “orthogonal” if the extent is over a threshold and “diagonal” if the extent is under a threshold, the threshold indicating a greater axis rotation over at least the full value of the lesser axis rotation (with thresholds that only slightly exceed a one-to-one match favoring the result of “diagonal” and higher thresholds making the result of “diagonal” increasingly selective). The front-or-back evaluation may analyze the positive or negative signs of transverse axis gyroscopic values during forward and backward swings (with the known local positive direction meaning positive values during a forward swing would indicate one orientation, and negative values during a forward swing would indicate the other). The left-or-right evaluation may analyze the positive or negative signs of perpendicular axis gyroscopic values during forward and backward swings (the known local positive direction meaning positive values during a forward swing would indicate one orientation, and negative values during a forward swing would indicate the other). Ultimately, the sensor assembly's roll may be estimated through combined orthogonal-or-diagonal, front-or-back, and left-or-right evaluations.

In the second calibration method, embodiments may sample motion data on the sensor hardware's axes to establish and analyze an axis hierarchy. (The sample may last for a certain duration, or may last until a threshold of motion data has been detected. Smaller samples may generate results more quickly, while larger samples may generate results with higher confidence.) Embodiments may use the sample to identify the axes as primary, secondary, and/or tertiary (as determined by greatest-to-least levels of sampled gyroscopic rotation for embodiments monitoring the activity with the gyroscope, or of sampled linear acceleration for embodiments monitoring the activity with the accelerometer). Embodiments may also set primary-secondary, primary-tertiary, and/or secondary-tertiary sign multipliers. Each sign multiplier may signify whether one of its hyphenated axes should have its values multiplied by −1 such that resulting values on both the hyphenated axes match in sign during performance of the intended activity, with the determination being to multiply by −1 if the positive and negative signs of values sampled on the axes differ more often than they match.

In the third calibration method, embodiments may determine whether or not to monitor the measurements on a given axis when tracking the intended activity. Embodiments may make this determination based on whether an axis meets a threshold of gyroscopic rotation and/or meets a threshold of linear acceleration. Embodiments also using the first and/or second calibration methods may determine whether or not to monitor an axis based on the results of those calibrations (e.g. determining if an axis is likely to detect motion in the performance of the intended activity based on its orientation, and/or based on its prominence in the axis hierarchy determined by a sample of motion data). Further embodiments may simply determine whether to monitor an axis based on developer preference and/or user input.

An elementary embodiment may use the third calibration method to select a single axis on which to track changes of direction, counting repetitions based on the number of those changes, and calculating cadence based on the frequency of those changes. While that offers a straightforward means of operation, alternative embodiments may enable additional features by monitoring data on multiple axes, and/or by tracking range-of-motion values for the swings. These embodiments may take data from multiple axes and use it to set each reading's interpreted gyroscopic value.

Embodiments may set the interpreted gyroscopic via a first gyroscopic interpretation method or a second gyroscopic interpretation method.

Embodiments using the first gyroscopic interpretation method may set the interpreted gyroscopic value as a sum of interpreted gyroscopic values from multiple axes, each axis value being “interpreted” in the sense of being potentially multiplied by −1 depending on the sensor assembly's orientation (as established by the first calibration method). This method may add together any combination of the gyroscopic values of the proximal, perpendicular, and transverse axes, with each axis value being potentially multiplied by −1 if so indicated by the sign multiplier for its axis (as set by the first calibration method). This addition may calculate an interpreted sum wherein a positive value measures degrees swung in a known direction relative to the user and a negative value measures degrees swung in the opposite direction relative to the user.

Embodiments using the second gyroscopic interpretation method may set the interpreted gyroscopic value as a sum of interpreted gyroscopic values from multiple axes, with each axis again being potentially multiplied by −1, this time depending on sample results from the second calibration method.

Some embodiments may set the second gyroscopic interpretation method's interpreted sum by adding together any combination of the direct gyroscopic value for the primary axis, the gyroscopic value for the secondary axis, potentially multiplied by −1 (if so indicated by the primary-secondary sign multiplier), and the gyroscopic value for the tertiary axis, potentially multiplied by −1 (if so indicated by the primary-secondary sign multiplier) This may calculate an interpreted gyroscopic sum wherein a positive value measures degrees swung in a direction causing positive gyroscopic values on the primary axis and a negative result measures degrees swung in a direction causing negative gyroscopic values on the primary axis.

Alternative embodiments may set the second gyroscopic interpretation method's interpreted sum by adding together any combination of the direct gyroscopic value for the secondary axis, the gyroscopic value for the primary axis, potentially multiplied by −1 (if so indicated by the primary-secondary sign multiplier), and the gyroscopic value for the tertiary axis potentially multiplied by −1 (if so indicated by the secondary-tertiary sign multiplier). This may calculate an interpreted gyroscopic sum wherein a positive value measures degrees swung in a direction causing positive gyroscopic values on the secondary axis and a negative result measures degrees swung in a direction causing negative gyroscopic values on the secondary axis.

Further alternative embodiments may set the second gyroscopic interpretation method's interpreted sum by adding together any combination of the direct gyroscopic value for the tertiary axis, the gyroscopic value for the primary axis, potentially multiplied by −1 (if so indicated by the primary-tertiary sign multiplier), and the gyroscopic value for the secondary axis, potentially multiplied by −1 (if so indicated by the secondary-tertiary sign multiplier). This may calculate an interpreted gyroscopic sum wherein a positive value measures degrees swung in a direction causing positive gyroscopic values on the tertiary axis and a negative result measures degrees swung in a direction causing negative gyroscopic values on the tertiary axis.

While the interpreted gyroscopic value may be used for measuring the motion of the intended activity, further embodiments may also use various methods for detecting motion distinct from the intended activity, and for early predictions of changes in the intended activity's speed.

Further embodiments may also use a detection method for identifying motion lateral to the intended activity. This may use one of two data comparisons depending on whether overall gyroscopic rotation during the intended activity is expected to be single-axis or multi-axis. (Rotation may be regarded as “single-axis” if gyroscopic readings from the intended activity are primarily concentrated on one axis, and “multi-axis” if gyroscopic readings from the intended activity are relatively balanced on at least two axes. The “single-axis” or “multi-axis” status may be determined by evaluations described below.)

Embodiments detecting lateral motion may determine whether the gyroscopic rotation from the intended activity is expected to be single-axis or multi-axis by checking whether past gyroscopic rotation on the primary axis exceeded a threshold greater than past gyroscopic rotation on the secondary axis (with thresholds that only slightly exceed a one-to-one match favoring the result of “single-axis” and higher thresholds making the result of “single-axis” increasingly selective). Alternatively embodiments with a designated proximal axis may determine the “single axis” or “multi axis” status by analyzing accelerometer readings on the two non-proximal axes, with the result of “single-axis” indicated by the absolute value of accelerometer readings on one non-proximal axis exceeding a threshold greater than the absolute value of accelerometer readings on the other non-proximal axis (with thresholds that only slightly exceed a one-to-one match favoring the result of “single-axis” and higher thresholds making the result of “single-axis” increasingly selective).

If rotation from the intended activity is expected to be single-axis, then embodiments detecting lateral motion may compare levels of current gyroscopic rotation on the primary and secondary axes, with lateral motion detected if the current secondary axis rotation exceeds a threshold relative to the current primary axis rotation (with higher thresholds resulting in more selective detection of lateral motion).

If rotation from the intended activity is expected to be multi-axis, then embodiments detecting lateral motion may compare the positive or negative signs of the addend values added together when calculating the interpreted gyroscopic value, with lateral motion detected if the signs of the addend values do not match.

Further embodiments may also use a detection method for identifying shakes as distinct from swing completions. This may include identifying individual, mid-swing shakes and/or identifying repetitive shaking motion.

Embodiments identifying individual, mid-swing shakes may establish a minimum range-of-motion requirement less than the greatest of the recent swing ranges (with lower requirements resulting in a smaller phase in which shakes can be detected). Embodiments may then check, when an interpreted gyroscopic sum indicates a change of rotational direction, whether the current swing's range-of-motion is under the minimum range-of-motion requirement. A change of rotational direction may be identified as a shake rather than a swing completion if the minimum range-of-motion requirement has not been met.

Embodiments identifying repetitive shaking motion may review recent swing ranges to see if a short-swing count threshold has been met regarding how many recent swing ranges have been under a short-swing range-of-motion threshold. The short-swing count threshold may be at least 2 recent swings, and the short-swing range-of-motion threshold may be less than the expected range-of-motion (with higher short-swing count thresholds and lower short-swing range-of-motion thresholds making detection more selective). Recently completed swings may be identified as repetitive shaking motion rather than performance of the intended activity if the short-swing count threshold has been met.

Further embodiments may also use a detection method for identifying sensor assembly disruptions (with the term “disruption” referring to a change in the sensor assembly's attachment status that interferes with the embodiment's general operation), such that sensor assembly disruptions are not mistaken for performance of the intended activity and may trigger software response. This may include identifying disruptions by a dual-value check (for both swing range-of-motion and acceleration), and/or identifying disruptions by a single-value check (for acceleration alone).

Embodiments detecting disruptions by the dual-value check may evaluate whether any current or recent swing's range-of-motion and a highest total acceleration (the term “total acceleration” meaning the sum of the absolute values of the measurements on each accelerometer axis in a reading) both exceed respective, moderate thresholds. The range-of-motion moderate threshold may be greater than the last completed swing's range-of-motion, and the total acceleration moderate threshold may be greater than the last completed swing's highest monitored total acceleration value (with higher values for both moderate thresholds making detection more selective). A sensor assembly disruption may be detected any time a swing's range-of-motion and highest total acceleration both exceed their respective, moderate thresholds.

Embodiments detecting disruptions by the dual-value check may evaluate whether a reading's total acceleration exceeds a severe threshold. The severe total acceleration threshold may be greater than the moderate total acceleration threshold if the dual-value check is also in use (with higher severe thresholds making detection more selective). A sensor assembly disruption may be detected any time a reading's total acceleration exceeds the severe threshold.

Further embodiments may also use a prediction method of anticipating changes in cadence before a new repetition duration, swing duration, or half-swing duration value is calculated. This may use varying checks to predict decreases or increases in cadence.

Embodiments predicting changes in cadence may predict a decrease in cadence by monitoring half-swing durations, swing durations, and/or repetition durations. A decrease in cadence may be predicted by an in-progress half-swing duration value exceeding the half-swing duration value that correlates with the most recently calculated cadence value, an in-progress swing duration value exceeding the swing duration value that correlates with the most recently calculated cadence value, and/or an in-progress repetition duration value exceeding the repetition duration value that correlates with the most recently calculated cadence value.

Embodiments predicting changes in cadence may predict an increase in cadence by monitoring swing rotation rates. An increase in cadence may be predicted any time an in-progress swing's rotation rate exceeds a threshold greater than the lowest rotation rate of recently completed swings (with higher thresholds making prediction more selective, and with the intensity of the increase predicted by the extent to which the in-progress rotation rate exceeds the threshold).

Embodiments may use a non-transitory, computer-readable storage medium having stored thereon a software program which, when executed by a processor, is operable for all functionality described above.

### DESCRIPTION OF THE DRAWINGS

FIG. 1 is a non-limiting example drawing depicting a use case for an embodiment using athletic activity as software input. 110 depicts a user seated on a stationary exercise bike with which the user may perform the athletic activity of pedaling while seated. 120 depicts a motion sensor assembly affixed to the user's thigh, which may swing in the user's anterior and posterior directions as the user pedals the bike. 130 depicts visual output of a software routine running on a processor with which the sensor assembly is interfaced; this example includes a readout of the user's pedaling RPM, which may be used to trigger software responses like setting the movement speed of a digital avatar.

FIG. 2 is a non-limiting example drawing depicting an alternative use case for an embodiment using athletic activity as software input. In this example, the user is on an elliptical machine with which the user may perform the athletic activity of pedaling while standing. This illustrates that the athletic activity does not need to be limited to one particular exercise or equipment type.

FIG. 3 is a non-limiting example drawing depicting a closeup of the sensor assembly's position on the user's ankle from FIG. 2. This illustrates that the sensor assembly does not need to be limited to one particular attachment position.

FIG. 4 is a non-limiting example drawing depicting an alternative use case for an embodiment using athletic activity as software input. In this example, the user is on an arm ergometer machine with which the user may perform the athletic activity of pedaling with the arms. This illustrates that the athletic activity does not need to be limited to one particular set of limbs.

FIG. 5 is a non-limiting example drawing depicting a closeup of the sensor assembly's position on the user's forearm from FIG. 4. This further illustrates that the sensor assembly does not need to be limited to one particular attachment position, but that it should be attached to a limb segment moved by the intended activity in repeating swings.

FIG. 6 is a non-limiting example drawing depicting examples of sensor assembly pitch. Given a seated user with a leg-mounted sensor assembly, 610 depicts a sensor assembly on the thigh whose pitch is angled, and 620 depicts a sensor assembly on the ankle whose pitch is upright.

FIG. 7 is a non-limiting example drawing depicting examples of sensor assembly roll about a limb segment's proximal-to-distal aspect. Given a seated user with a thigh-mounted sensor assembly, 710 depicts a sensor assembly whose roll is front-facing, and 720 depicts a sensor assembly whose roll is side-facing.

FIG. 8 is a non-limiting example drawing depicting context for an intended activity. In this example, a user is seated on a stationary exercise bike, and the intended activity is to pedal the bike.

FIG. 9 is a non-limiting example drawing depicting the type of motion that may be detected during performance of the intended activity established in FIG. 8. This motion moves the sensor assembly in repeating swings in the user's anterior and posterior directions, which should be the basis for calculating statistics about the intended activity.

FIG. 10 is a non-limiting example drawing depicting a type of motion lateral to the intended activity established in FIG. 8. This motion moves the sensor assembly in swings to the user's left and right directions, which preferred embodiments may identify and not mistake for performance of the intended activity. Alternative embodiments could also treat this type of motion as the intended activity instead (e.g. for counting reps and/or cadence of hip abduction/adduction motions).

FIG. 11 is a non-limiting example drawing depicting swing phases that may be detected for leg-pedaling motion with a sensor assembly worn on the thigh. For each complete rotation, the sensor assembly at this placement detects a down-swing (when the knee moves downward) and an up-swing (when the knee moves upward). 1110 depicts the down-swing phase, and 1120 depicts the up-swing phase.

FIG. 12 is a non-limiting example drawing depicting swing phases that may be detected for leg-pedaling motion with a sensor assembly worn on the ankle. For each complete rotation, the sensor assembly at this placement detects a front-swing (when the foot moves forward) and a back-swing (when the foot moves back). 1210 depicts the front-swing phase. 1220 depicts the back-swing phase, and also shows that the back-swing has a low mid-point in which the sensor assembly goes through a downward-to-upward direction change, which is relevant to roll estimation at an upright pitch.

It is notable the transition points for the phases in FIGS. 11 and 12 are not the same. This is because the different swing-type extremes are not simultaneous—the knee reaches its highest point in the middle of the foot's forward motion, and its lowest point in the middle of the foot's backward motion. Thus, FIGS. 11 and 12 together illustrate that a given activity (e.g. pedaling a bike) could be detected in different swing types (e.g. up/down or front/back) for different sensor assembly positions.

FIG. 13 is a non-limiting example drawing depicting swing phases that may be detected for arm-pedaling motion with a sensor assembly worn on the wrist. For each complete rotation, the sensor assembly at this placement detects a down-swing (when the wrist moves downward) and an up-swing (when the wrist moves up). 1310 depicts the down-swing, and 1320 depicts the up-swing. It is also notable that phases show the limb segment's proximal direction is not always “upward” (as in running against the pull of gravity). This is especially evident at the high point of each phase (at the start of the down-swing, and at the end of the up-swing), where the forearm's proximal direction from the wrist to the elbow goes downward. This may be relevant for embodiments that ascertain the local positive direction of the sensor assembly's proximal axis.

FIG. 14-17 are non-limiting example drawings depicting a variety of axis orientations for a sensor assembly affixed to the front of a user's thigh. Embodiments may be configured to work with any of these orientations.

FIG. 14 is a non-limiting example drawing depicting a thigh-mounted sensor assembly whose axis orientations might be considered standard for some embodiments. The axes here show the first calibration method's designations of the proximal axis, perpendicular axis, and transverse axis. 1410 depicts the proximal axis, which in this configuration is the X axis for the example hardware. 1420 depicts the perpendicular axis, which in this configuration is the Z axis for the example hardware. 1430 depicts the transverse axis, which in this configuration is the Y axis for the example hardware. As the following figures show, alternative embodiments may use different axis orientations.

FIG. 15 is a non-limiting example drawing depicting a thigh-mounted sensor assembly whose orientation is tilted 90° as compared to the FIG. 14 example. 1510 depicts the proximal axis, but given the hardware's orientation, this is now the Y axis for the example hardware. 1520 depicts the perpendicular axis, which is still the Z axis for the example hardware. 1530 depicts the transverse axis, which is now the X axis for the example hardware.

FIG. 16 is a non-limiting example drawing depicting a thigh-mounted sensor assembly whose orientation is tilted 180° as compared to the FIG. 14 example. 1610 depicts the proximal axis, which is again the X axis for the example hardware, but compared to the FIG. 14 example, a local positive direction on this axis is now inverted. 1620 depicts the perpendicular axis, which is still the Z axis for the example hardware with the same local positive direction as in the FIG. 14 example. 1630 depicts the transverse axis, which is again the Y axis for the example hardware, but compared to the FIG. 14 example, a local positive direction on this axis has also been inverted.

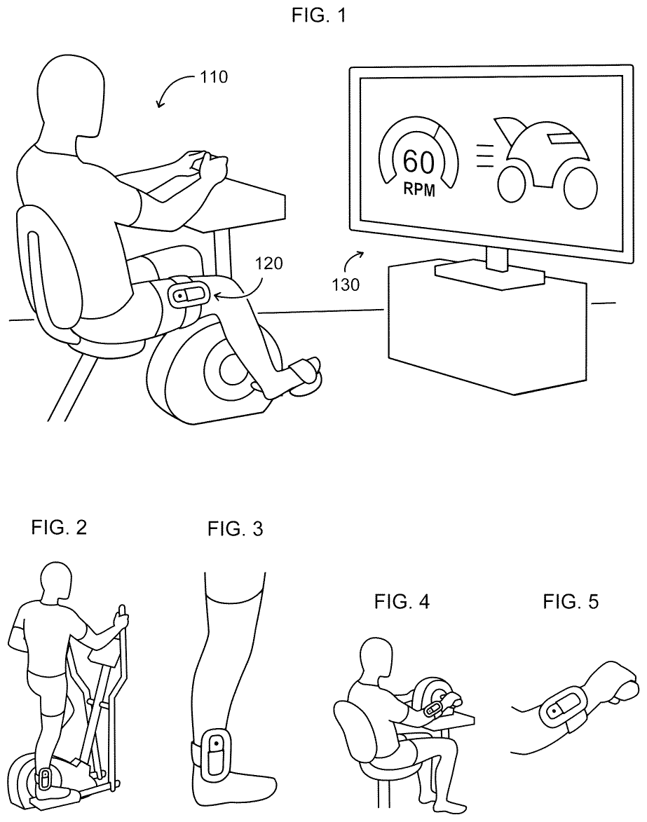

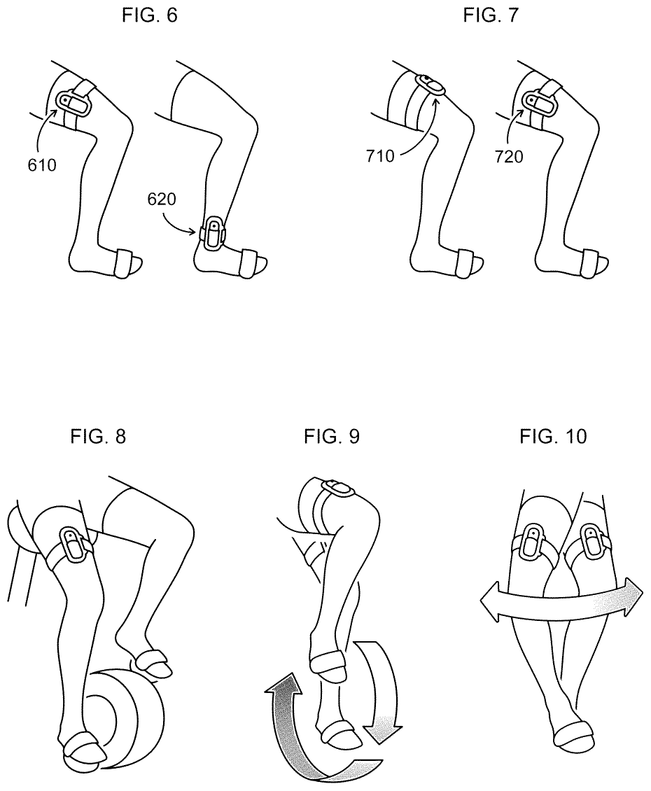

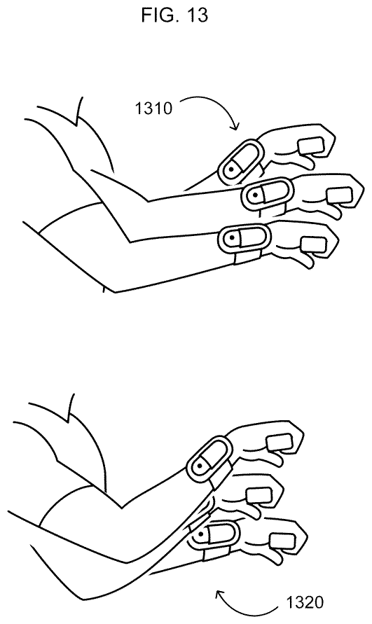

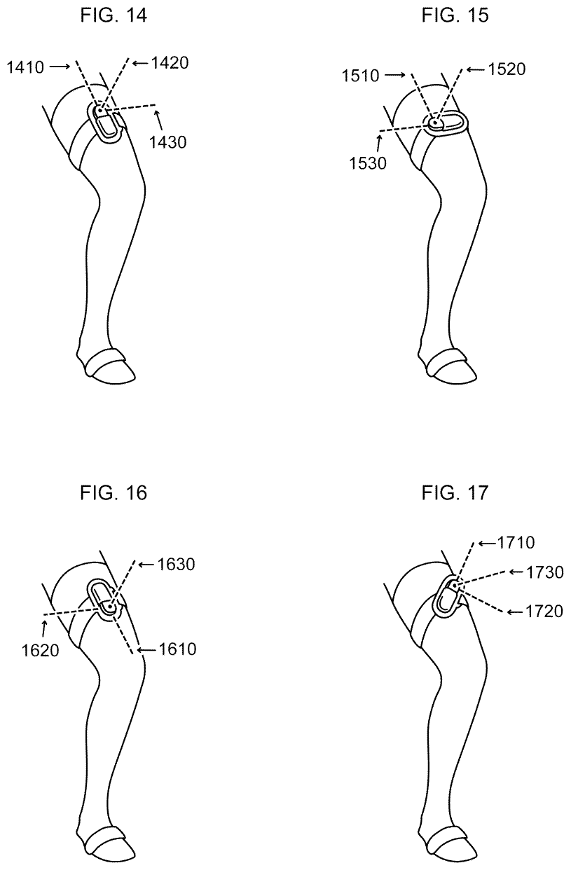

Embodiments may be configured to work with any of the sensor assembly orientations depicted in FIG. 14-16. If embodiments use the first calibration method to ascertain the sensor assembly's orientation, then the pitch and roll estimations may rely on known data points for which axes are designated proximal, perpendicular, and transverse, and how the local positive directions for each axis are oriented relative to the limb segment. (If an embodiment's sensor assembly includes accelerometer hardware and the embodiment expects the limb segment's proximal direction to go “up” against the pull of gravity, as would usually be the case during leg-pedaling exercises, then a local positive direction on the proximal axis may also be estimated by whether the pull of gravity on the proximal axis results in a positive or negative accelerometer reading. However, as shown in FIG. 13, it is not always a given that the limb segment's proximal axis will go “up.”) Alternatively, if embodiments use the second calibration method to identify the axes as primary, secondary, and tertiary, then full functionality may be possible with the sensor assembly at any of these positions without estimating orientation.

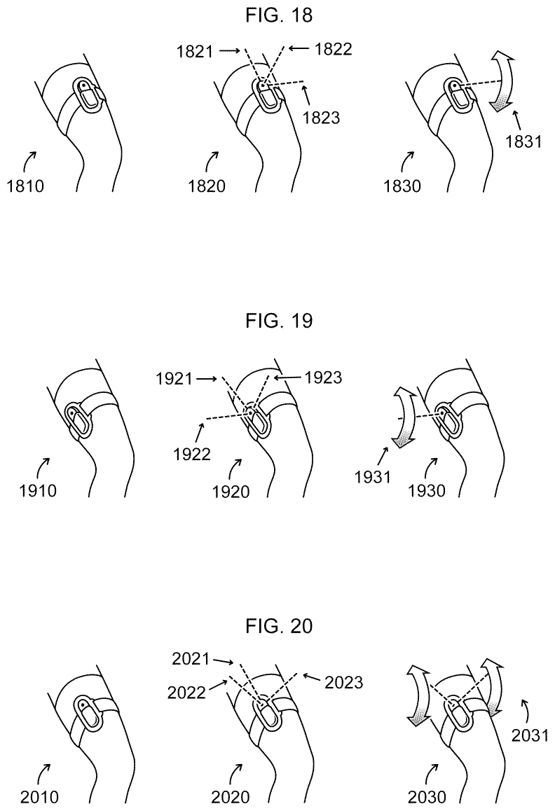

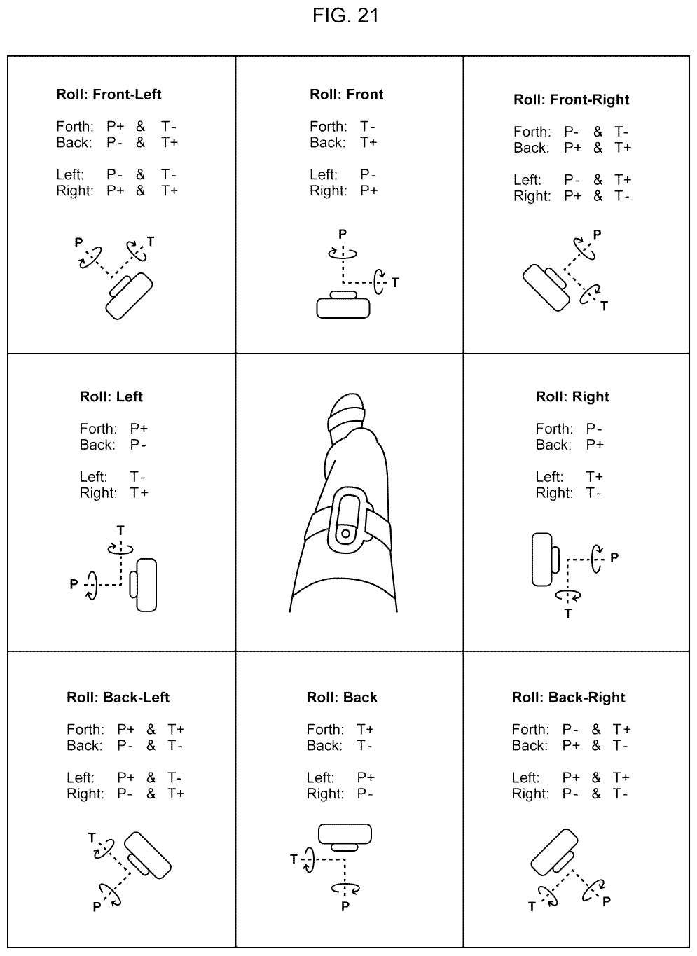

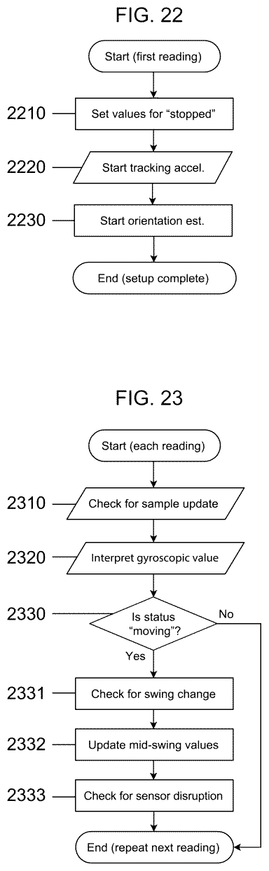

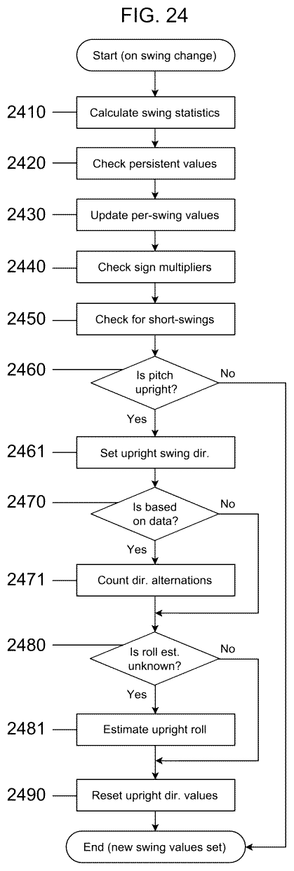

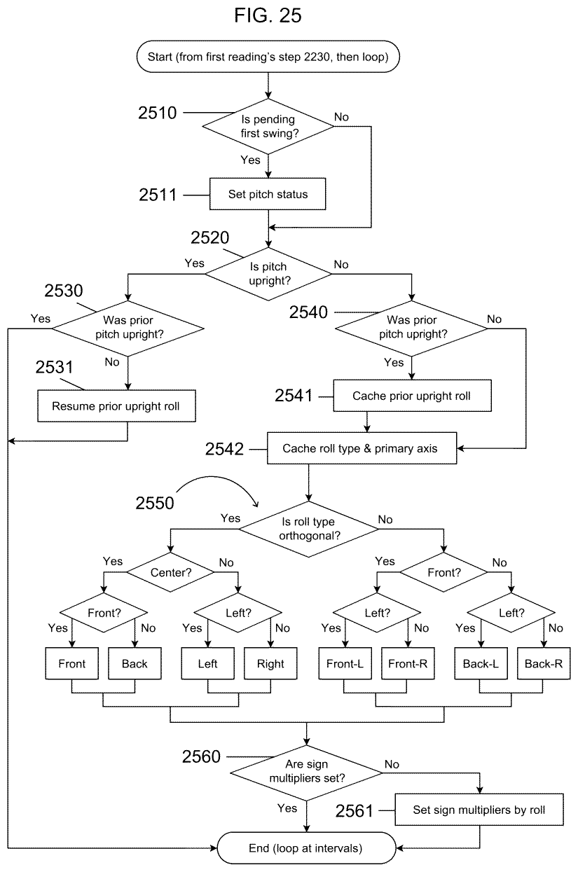

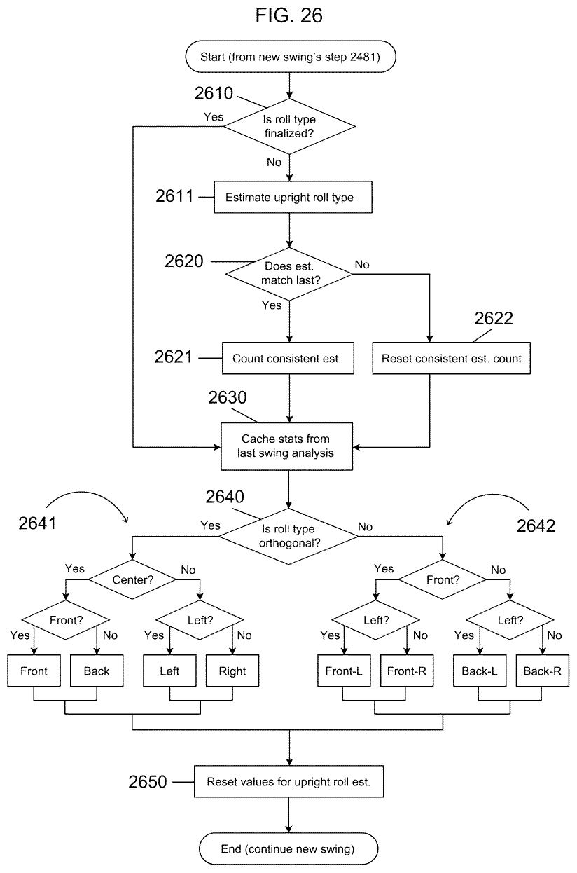

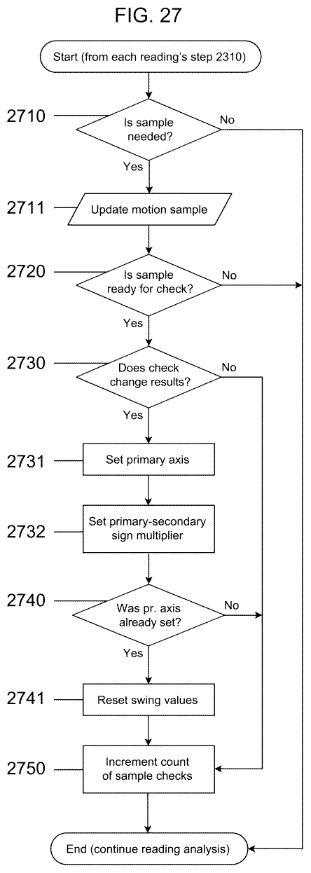

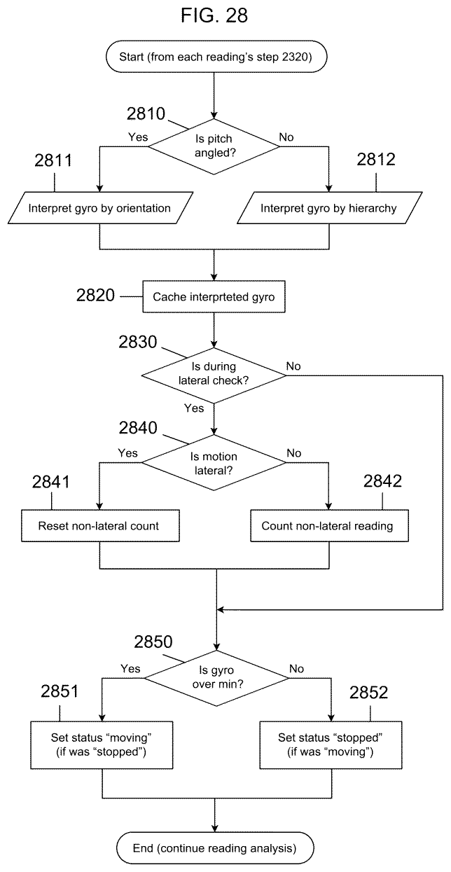

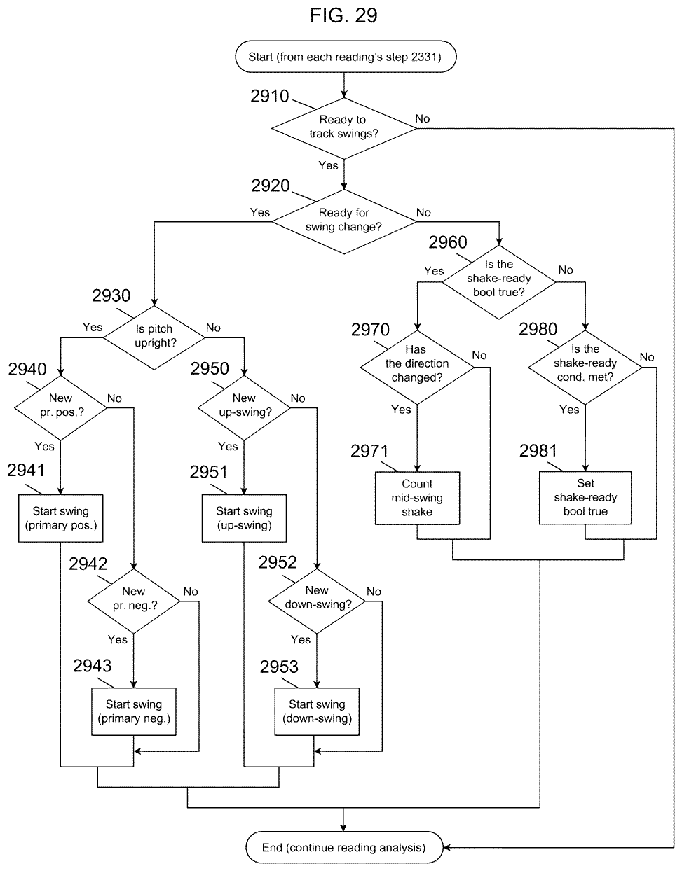

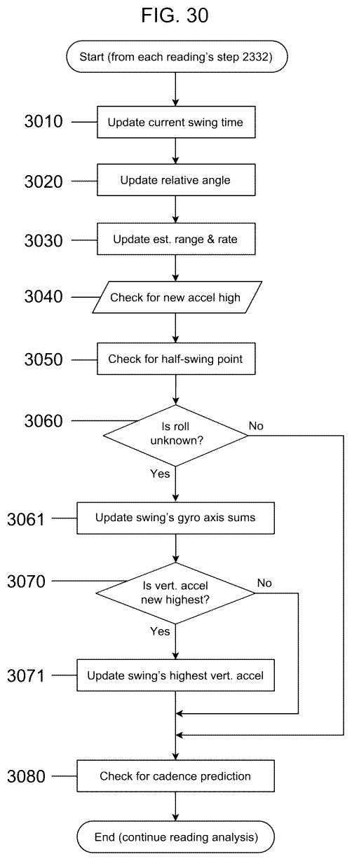

FIG. 17 is a non-limiting example drawing depicting a thigh-mounted sensor assembly whose orientation is tilted at a 45° angle as compared to the FIG. 14 example. 1710 depicts the example sensor hardware's X axis, which does not align with any of the first calibration method's proximal, perpendicular, or transverse axis designations. 1720 depicts the example sensor hardware's Z axis, which still aligns with the first calibration method's perpendicular axis designations. 1730 depicts the example sensor hardware's Y axis, which does not align with any of the first calibration method's proximal, perpendicular, or transverse axis designations. Since this example does not have an axis aligned with the limb segment's proximal-to-distal axial dimension, it may not be well suited to the first calibration method's roll estimation. However, an embodiment using this sensor assembly orientation could still ascertain roll via developer-intended guidelines or user-reported indicators, or it could use the second calibration method or third calibration method and operate without estimating orientation.

FIG. 18-20 are non-limiting example drawings depicting various rolls about the proximal axis for a sensor assembly affixed to a user's thigh. These figures also depict orientations of the proximal, perpendicular, and transverse axes given each roll, and they depict which axes would measure gyroscopic rotation during pedaling motion for each sensor assembly position.

FIG. 18 is a non-limiting example drawing depicting a sensor assembly affixed on a user's thigh with a front-facing roll about the proximal axis. 1810 depicts this sensor assembly position as an unobstructed view without any representation of the axis designations. 1820 adds indicators for the axis designations, with 1821 depicting the proximal axis, 1822 depicting the perpendicular axis, and 1823 depicting the transverse axis. 1830 focuses on the axis about which the sensor assembly would rotate during performance of the intended leg-pedaling activity, with 1831 showing this would be the transverse axis given a front-facing roll.

FIG. 19 is a non-limiting example drawing depicting a sensor assembly affixed on a user's thigh with a side-facing roll about the proximal axis. 1910 depicts this sensor assembly position as an unobstructed view without any representation of the axis designations. 1920 adds indicators for the axis designations, with 1921 depicting the proximal axis, 1922 depicting the perpendicular axis, and 1923 depicting the transverse axis. 1930 focuses on the axis about which the sensor assembly would rotate during performance of the intended leg-pedaling activity, with 1931 showing this would be the perpendicular axis given a side-facing roll.

FIG. 20 is a non-limiting example drawing depicting a sensor assembly affixed on a user's thigh with a roll about the proximal axis diagonally between front-facing and side-facing. 2010 depicts this sensor assembly position as an unobstructed view without any representation of the axis designations. 2020 adds indicators for the axis designations, with 2021 depicting the proximal axis, 2022 depicting the perpendicular axis, and 2023 depicting the transverse axis. 2030 focuses on the axes that would detect gyroscopic rotation from performance of the intended leg-pedaling activity, with 2031 showing this would be both the transverse and perpendicular axes given a diagonal roll.

FIG. 21 is a non-limiting example chart depicting how an example sensor assembly's local positive and negative gyroscopic values on the perpendicular and transverse may correspond to directions of limb motion at various rolls about the proximal axes. (For this example hardware, the arrows around each axis diagram indicate directions of local positive gyroscopic rotation for the axes.) It is notable that if a sensor assembly's roll changes, the meanings of positive and negative signs for gyroscopic readings on each axis may change as well. This illustrates the potential utility of roll estimation and sign comparisons for distinguishing one swing's direction from another, and for identifying whether motion aligns with an intended activity or is lateral to it (as depicted in FIG. 8-10).

FIG. 22 is a non-limiting example flowchart depicting logic that may be executed when the method receives its first reading from the motion sensor assembly (e.g. upon loading a training program or video game level). Step 2230 is further expanded in FIG. 25.

FIG. 23 is a non-limiting example flowchart depicting logic that may be executed by the method each reading. The steps 2310, 2320, 2331, and 2332 are further expanded in FIGS. 27-30.

FIG. 24 is a non-limiting example flowchart depicting logic that may be executed each time a new swing is detected. This may be called by step 2331 in FIG. 23, and step 2481 is further expanded in FIG. 26.

FIG. 25 is a non-limiting example flowchart depicting logic that may be executed for the first calibration method to estimate the sensor assembly's pitch, and if the pitch is determined to be angled, to also estimate the sensor assembly's roll about the proximal axis. This logic may initially be called by step 2230 in FIG. 22, and it may then repeat at regular intervals to check for changes in orientation (e.g. to maintain functionality if a sensor assembly slides from the front of a user's thigh to the side). It may be acceptable for these ongoing checks to be less responsive than the logic in FIG. 23 without compromising user experience, so the execution frequency here may be lower to reduce computing demands (e.g. FIG. 23 may execute 60 times per second for high responsiveness, but FIG. 25 may execute at a lower rate, e.g. 10 times per second, though alternate embodiments may use other intervals).

FIG. 26 is a non-limiting example flowchart depicting logic that may be executed for the first calibration method to estimate the sensor assembly's roll about the proximal axis if the pitch has been determined to be upright. This may be called by step 2471 in FIG. 24 (i.e. each time a new swing is detected, conditional upon the sensor assembly's pitch being upright).

FIG. 27 is a non-limiting example flowchart depicting logic that may be executed for conducting the second calibration method's sample if called for. This may be called by step 2310 in FIG. 23.

FIG. 28 is a non-limiting example flowchart depicting logic that may be executed for setting the current reading's interpreted gyroscopic value. This may be called by step 2320 in FIG. 23.

FIG. 29 is a non-limiting example flowchart depicting logic that may be executed for detecting a swing change. This may be called by step 2331 in FIG. 23.

FIG. 30 is a non-limiting example flowchart depicting logic that may be executed for updating mid-swing values. This may be called by step 2332 in FIG. 23.

### DESCRIPTION OF THE INVENTION

The following description explains an embodiment intended to calculate repetitions and cadence of a pedaling motion of the arms or legs for use as software input. It is noted that alternative embodiments may be intended to work with different activities.

This embodiment has been prototyped with a sensor assembly that includes a gyroscope and an accelerometer. The embodiment assumes use of developer-intended guidelines for orienting the sensor assembly such that the local positive direction of linear acceleration on the proximal axis runs proximally along the limb segment, and the local positive direction of linear acceleration on the perpendicular axis runs perpendicularly outward from the sensor assembly away from the limb segment. With these guidelines followed, the sensor assembly may be placed on the thigh or the ankle for leg-pedaling, or on the forearm or upper arm for arm-pedaling, at any roll about the chosen limb segment's proximal-to-distal axial dimension. The local positive directions of gyroscopic rotation will then be oriented by the sensor assembly's roll as illustrated in FIG. 21. This embodiment is fully operable without requesting any indicators of sensor assembly orientation from the user. It is noted that alternative embodiments may use different approaches to the sensor assembly's orientation.

This embodiment uses the first calibration method to estimate if the sensor assembly's pitch is deemed upright or angled, and further uses the first calibration method to estimate roll if the pitch is angled, otherwise using the second calibration method if the pitch is upright. Based on the guidelines for sensor assembly orientation, for this embodiment, the third calibration method is only present in the developer's decision to not monitor proximal axis gyroscopic rotations when tracking swings of the intended activity. It is noted alternative embodiments may use the calibration methods in different ways.

This embodiment is intended to track activity types involving limb segment swings in the user's anterior and posterior directions. This embodiment labels these swings as “up-swings” and “down-swings” for angled limb segments (e.g. the thigh during leg-pedaling, or any segment of the arm during arm-pedaling) and “front swings” and “back swings” for upright limb segments (e.g. the shin during leg-pedaling). It is noted that alternative embodiments may label swings differently, and/or track swings in different directions relative to the user (e.g. hip adductions and abductions, as illustrated in FIG. 10).

This embodiment calculates an interpreted gyroscopic value for each reading (using the first gyroscopic interpretation method if the sensor assembly's pitch is angled, and the second gyroscopic interpretation method if the sensor assembly's pitch is upright), which facilitates tracking swing transitions as well as detecting various types of unexpected motion. It is noted that alternative embodiments could track swing transitions using a variety of other detection methods described above.

This embodiment sets sign multipliers as numeric values of 1 or −1; when calculating a reading's interpreted gyroscopic value, it multiplies each of the gyroscopic values from the non-proximal axes by their respective sign multiplier, resulting in a value being multiplied by −1 if called for by how the sign multiplier was determined. It is noted that alternative embodiments could take different approaches to the sign multipliers for similar results (e.g. setting each sign multipliers as a boolean values for whether or not its axis should have its values multiplied by −1).

Furthermore, all example values for thresholds, requirements, etc. have been tested and found effective for this embodiment, but alternative embodiments may use different values depending on their intended activities, variations in equipment, and developer preference.

It is thus appreciated that understanding the embodiment described herein would facilitate alternative implementations that, while not explicitly described, employ the disclosure's principles and fall within its scope.

### First Reading

In this embodiment, when the software first receives a reading from the sensor assembly, the initialization steps depicted in FIG. 22 are executed. This happens when the embodiment begins to operate, and it may later repeat for contextual recalibration (e.g. when pausing and un-pausing the software, in case the user has changed equipment or sensor assembly position). The setup steps are as follows:

In step 2210, this embodiment sets values for multiple variables to their “Stopped” status. (The logic for setting these values may also be called again if the sensor assembly's status later transitions from “moving” back to “stopped,” as checked for in step 2320.) These values include status-check variables (e.g. booleans, enumerators, etc. depending on developer preference) for the sensor assembly's status as “stopped” or “moving” (initially “stopped”), as well as for its status of being in any of the possible swing types (up- or down-swing if the pitch is angled, front- or back-swing if the pitch is upright, all initially false). This step also zeroes-out numeric values for the current cadence, swing duration, pedaling confidence (see step 2410), mid-swing shakes counted in the current swing (see step 2971), and consecutive readings with non-lateral motion (see step 2830). This step also resets “per-swing values” to their defaults (which may be assigned by developer preference) for the gyroscopic motion threshold for the “moving” status, and the duration checked for it to remain under that threshold to revert to the “stopped” status. This step also sets the values for highest and lowest relative angles in the current swing to the current value for the relative angle (starting at a reference value of 0 when the software first initializes, and putting the highest and lowest values at an equal starting point to later update when a swing is underway, with the updated values then used to determine the swing's range-of-motion). For contextual recalibrations, if the sensor assembly's pitch is upright, this step sets its estimated direction of motion to “Unknown” and zeroes-out the count of alternating direction estimations, and it resets the values for roll estimation if the roll is not yet finalized.

In step 2220, this embodiment begins tracking accelerometer values from the sensor assembly with a process that loops in timed intervals (e.g. 10 times per second, though alternate embodiments may use other intervals). This caches the accelerometer values from the two non-proximal axes. Once cached, each axis has its recent values averaged (e.g. the values cached over the last full second); doing so smooths the signal's response to varying user motion, with the smoothed values then indicating gravity's consistent effect on the accelerometer, which may then be used by the first calibration method to estimate roll if the sensor assembly's pitch is angled (as depicted in FIG. 25, and described in detail further below). The proximal axis, by contrast, may have its accelerometer values monitored for vertical acceleration during user motion; therefore it may be checked more frequently and smoothed to a lesser degree (as depicted in FIG. 30, and described in detail further below).

In step 2230, this embodiment begins checking if the first calibration method should estimate roll about the proximal axis for the sensor assembly at an angled pitch. The logic for this is expanded in FIG. 25 and described in detail further below.

### Each Reading

In each reading of this embodiment's operation, the software executes the logic depicted in FIG. 23. For optimum responsiveness, it is preferred for this to execute as frequently as the sensor assembly is able to provide new motion data, though longer and/or varying intervals may be used (depending on computing power, developer preference, etc.). For the purposes of the method's description, the term “each reading” may refer to any rate of execution with new motion data available.

Steps 2310 and 2320 are executed in each reading of the embodiment, while steps 2331, 2332, and 2323 are conditional up on the embodiment's decision 2330 returning true.

In step 2310, this embodiment checks if the second calibration method calls for a motion sample update. This proceeds if the sensor assembly's pitch is upright and its roll is not yet estimated; in this case, motion data is briefly sampled, and analysis of the sample allows the embodiment to start tracking the activity provisionally before the sensor assembly's roll is known. The logic for this is expanded in FIG. 27 and described in detail further below.

In step 2320, this embodiment sets the interpreted gyroscopic value based on gyroscopic data from the sensor assembly. This step determines the extent to which any detected motion aligns with the activity intended to be tracked, and it updates the sensor assembly's status as “moving” or “stopped” if called for. The logic for this is expanded in FIG. 28 and described in detail further below.

In decision 2330, this embodiment checks the sensor assembly's “moving”/“stopped” status as set by step 2320. The remaining steps 2331 through 2333 execute if the status is “moving.”

In step 2331, this embodiment checks the interpreted gyroscopic value from step 2320 to determine if a swing change is detected. This step may also include shake detection for a “mid-swing shake,” referring to a change in the sensor assembly's direction of motion before a swing is completed (e.g. from muscular tremors or rattling equipment). The logic for detecting swing changes and mid-swing shakes is expanded in FIG. 29 and described in detail further below.

In step 2332, this embodiment updates mid-swing values (data for the swing currently underway). These values include the swing duration, current relative angle, estimated range-of-motion (in degrees rotated), rotation rate (in degrees per second), and highest accelerometer activity. If the sensor assembly's roll is unknown (as may be the case if its pitch is upright, and the motion analysis is not yet complete), this step may also cache additional values for analysis and roll estimation. The logic for updating all of this is expanded in FIG. 30 and described in detail further below.

In step 2333, this embodiment checks for a sensor disruption. This check evaluates the swing's range-of-motion and total acceleration (the sum of the absolute values of the measurements on each accelerometer axis in a reading) to detect unexpected motion (e.g. the sensor assembly falling out of position), which may trigger a software response (e.g. a pause in tracking and/or a message being displayed to the user). The disruption detection uses two checks: a single-value check for whether a swing's range-of-motion and a highest total acceleration both exceed moderate thresholds, and a single-value check for whether the current total acceleration exceeds a severe threshold. Each threshold is updated dynamically upon each swing completion, and the threshold calculations are described in step 2430.

### Each New Swing

Each time this embodiment detects a transition from one swing to another (as checked for in step 2331), the embodiment executes the logic depicted in FIG. 24. In this embodiment, steps 2410, 2420, 2430, 2440, and 2450 are executed for each new swing, while the rest of the steps in FIG. 24 are conditional upon checks regarding the sensor assembly's orientation.

In step 2410, this embodiment calculates swing statistics, comprising multiple variables cached and/or reset in light of the latest swing completion. These include the following, with details in the paragraphs below:
Repetition count
Cadence
Completed swing range-of-motion
Recent swing ranges-of-motion
Previous swing duration (full or half)
Previous swing rotation rate
Recent swing rotation rates
Previous swing highest total acceleration
Last two swings' highest vertical accelerations (if pitch is upright)
Mid-swing shake count
Pedaling confidence

The repetition count is the total number of times the tracked activity has been repeated (e.g. the number of “rotations” when tracking a pedaling motion). For this embodiment, each repetition of the intended pedaling motion comprises two full swings (an up-swing and a down-swing, or a front-swing and a back-swing, depending on the sensor assembly's position). Therefore the repetition count increments by 0.5 if tracking full swings, or 0.25 if tracking half-swings (more information on half-swing tracking is explained in the description of step 2430 below).

The cadence is the rate of repetitions indicated by the completed swing's duration. Cadence may be calculated as a unit of time numerator divided by repetition duration, or a unit of time numerator divided by the number of swings per repetition, then further divided by swing duration, or half a unit of time numerator divided by the number of swings per repetition, then further divided by half-swing duration. This embodiment sets this value as RPM (rotations per minute); therefore it calculates cadence as 30 divided by the full swing duration, or 15 divided by the half-swing duration if tracking half-swings. (Multiple RPM values may be cached and averaged as swings continue. Such averaging can increase smoothness, but it may decrease responsiveness to changes in cadence; to counter this, the duration and degrees-per-second values described below may be used for predicting cadence changes, as described in step 3080.)

The completed swing range-of-motion is the total estimated degrees that the sensor assembly rotated during the completed swing. This can be cached directly from the “mid-swing values” as described in step 3030. Once the most recent range-of-motion is cached, step 2410 also sets the sensor assembly's current relative angle as both the highest and lowest angles so far in the new swing (these are subject to updates during the swing to estimate its rotational range).

Recent swing ranges-of-motion, including that of the just-completed swing, are cached for setting various range expectations and thresholds throughout the embodiment. This embodiment uses an array with 4 elements to cache swing ranges-of-motion from the last 2 full rotations (though alternative embodiments may cache more or fewer recent ranges-of-motion).

The previous swing duration is the length of time the finished swing took to complete. This may be cached as a half-swing duration if half-swings are being tracked (with more detail on half-swing timing in step 3050); otherwise a full swing duration may be cached instead. The previous duration value may be used for predicting cadence changes, particularly for predicting a decrease before the next cadence value is ready (as described in step 3080).

The previous swing rotation rate is the completed swing's overall rate of rotation, calculated in degrees-per-second as the swing's total range-of-motion divided by its total duration.

Recent swing rotation rates, including that of the just-completed swing, are also cached collectively; these values may be used for predicting cadence changes in step 3080. This embodiment uses an array with 4 elements to cache rotation rates from the last 2 full rotations (though alternative embodiments may cache more or fewer recent ranges-of-motion).

The previous swing highest total acceleration is the sum of the absolute values on all accelerometer axes from the strongest accelerometer reading detected during the completed swing. (This can be cached directly from the mid-swing values as described in step 3040.) This embodiment uses this value as a basis for thresholds to be set in step 2420, which may be used for the detection of sensor disruptions as described in step 2333.

The last two swings' highest vertical accelerations (cached by this embodiment if the sensor assembly's pitch is upright) consist of the highest vertical acceleration value cached in the just-completed swing, and the highest vertical acceleration value cached in the swing before that. (For this embodiment, as mentioned in step 2220′s description, “vertical acceleration” is measured by accelerometer values on the proximal axis. Each swing's highest vertical acceleration is cached in step 3071.) These values may be used for upright swing direction estimation in step 2461.

If any mid-swing shakes were counted during the previous swing, the “shakes in current swing” value is reset to zero for the new swing.

The pedaling confidence is a value rating this embodiment's perceived likelihood that the user is performing the intended motion (i.e. the pedaling motion tracked by this embodiment, as opposed to swaying or shaking of the user's leg). For this embodiment, this value starts at 0 before the embodiment has detected motion (indicating no confidence the user is pedaling), then rises by 1 with each successful swing completion, until reaching this embodiment's maximum value of 4 (indicating high confidence the user is pedaling). The confidence value facilitates checking the user's stage of progression from static to steady pedaling; this is checked within the current embodiment (e.g. in decision 2510), and it may also be checked by software using the embodiment (e.g. for setting a digital avatar's rate of acceleration when the user begins to pedal).

In step 2420, this embodiment checks for whether to set persistent values, comprising values intended to be cached once per activity session. (Once set, these may be left alone until a context calls for recalibration, e.g. if the embodiment is paused and un-paused, which may indicate the user has changed position, equipment, etc.). This embodiment sets these values the first time enough completed swing ranges have been cached to calculate an expected range-of-motion going forward (e.g. once four full swings have been cached, their ranges-of-motion represent the degrees covered by swings in the last two full pedaling rotations). The persistent values include the following, with details in the paragraphs below:
Full swing expected range-of-motion
Half-swing expected range-of-motion
Short-swing threshold range-of-motion

The full swing expected range-of-motion is an estimated value for how many degrees each full swing is expected to rotate. This can be calculated by averaging the ranges-of-motion of recently cached ranges (e.g. from four full swings, as described above). To maximize flexibility (e.g. for shifts in the user's posture, and/or irregularities in gyroscopic readings), this embodiment treats the expected range-of-motion value as a general estimate rather than a strict requirement. This serves as the basis for the next two values.

The half-swing expected range-of-motion (calculated as one half of the full swing expected range-of-motion) is the degree value at which a current swing is estimated to have passed its half-way point. This is used for tracking half-swings (as described further in step 3050).

The short-swing threshold range-of-motion, which may be used for shake detection, is the range-of-motion value under which completed swings may be deemed “short,” with multiple short-swings in a row indicating a repetitive shaking motion rather than the intended pedaling motion. For adaptability to various equipment sizes, this embodiment calculates the short-swing threshold as 10° under 75% of the full swing expected range-of-motion with a minimum value of 2.5° (though alternate embodiments may use other threshold calculations). Further detail on this value's role is provided in the description for step 2450 below.

In step 2430, this embodiment sets per-swing values, comprising multiple dynamic thresholds for status changes (e.g. for toggling whether a swing is ready to complete, or toggling status between “moving” and “stopped,” with the threshold values being dynamic in that they update with each swing completion). These include the following, with details in the paragraphs below:
Whether to track half-swings
Swing minimum range-of-motion requirement
Gyroscopic threshold for “moving”
Wait-time threshold for “stopped”
Sensor disruption thresholds

The setting for whether to track half swings determines if the embodiment will calculate a new cadence value at the new swing's mid-point as opposed to waiting for the new swing's completion. Half-swing tracking can improve responsiveness at low RPM (when times between cadence updates derived from full swings are relatively long), but it can also introduce unwanted “noise” at high RPM (when times between cadence updates derived from full swings are relatively short). Therefore, this embodiment determines whether to track half-swings based the cadence set in step 2410, with the result opting to track half-swings on one side of a threshold (e.g. for cadence under 120 RPM, meaning swing durations over 0.25 seconds, though alternative embodiments may use other thresholds), and to not track half-swings on the other side of that threshold (e.g. for cadence of 120 RPM or higher, meaning swing durations at or under 0.25 seconds, though alternative embodiments may use other thresholds). (This evaluation also opts to not track half-swings if the half-swing expected range-of-motion value has not yet been set.)

The swing minimum range-of-motion requirement, which may be used for shake detection, is the minimum range-of-motion required for identifying a gyroscopic direction change as a swing completion rather than an individual, mid-swing shake. To maximize compatibility with athletic equipment of various sizes (e.g. including mini-pedalers, the use of which may rotate the sensor assembly through just a small range-of-motion), this minimum range-of-motion requirement starts relatively low by default (e.g. 3°). Each time a swing completes, the requirement updates based on the cached ranges of completed swings. To prioritize flexibility for variability between swings, this requirement is calculated as 10° under the lowest recently cached range-of-motion for a completed swing, with a minimum value of 1° (though alternative embodiments may use other threshold calculations). This embodiment prioritizes flexibility for equipment size and sensor assembly placement, but other embodiments may calculate the requirement more strictly to more selectively differentiate full swings from shakes (e.g. other embodiments may require full-size athletic equipment, and/or may ask the user to select the size of their equipment, in which case the range-of-motion requirement may be more strict.)

The gyroscopic threshold for “moving” is the minimum interpreted gyroscopic absolute value checked for in step 2320 to maintain the sensor assembly's status as “moving.” A single, constant threshold for this may not be suitable at all speeds: a relatively high threshold may be oversensitive at low speed (e.g. a threshold of 0.1 may incorrectly trigger the “stopped” status while the user is pedaling slowly), while a relatively low threshold may have insufficient sensitivity at high speed (e.g. a threshold of 0.05 may cause lag before triggering the “stopped” status if the user stops after pedaling quickly). Therefore, this embodiment adapts the gyroscopic threshold for “moving” to vary by cadence, interpolating from a high value to a low value by RPM (e.g. this embodiment interpolates from 0.1 for 10 RPM to 0.05 for 200 RPM, though alternative embodiments may use other thresholds) each time a new cadence value is calculated.

The wait time for “stopped” is a duration variable checked for in step 2320 when detecting if the user's motion comes to a stop; if the interpreted gyroscopic value's absolute value stays under gyroscopic threshold for “moving” for this duration, then the status changes from “moving” to “stopped.” As with the gyroscopic threshold for “moving,” a single, constant threshold for this may not be suitable at all speeds: it may be preferable to check for a longer duration at low speed (to avoid false-positive stops for very gradual motion), and a shorter duration at high speed (to limit latency of responses to actual stops). Therefore, this embodiment adapts the wait-time threshold for “stopping” to also interpolate from high to low value by RPM (e.g. this embodiment interpolates from 0.75 seconds for 10 RPM to 0.3 seconds for 200 RPM, though alternative embodiments may use other thresholds).

The sensor disruption thresholds are the limits checked for when detecting unexpected motion (e.g. the sensor assembly falling out of position) as explained in the description for step 2333. This embodiment uses two moderate thresholds for a dual-value check (which evaluates both total acceleration and swing range-of-motion), and one severe threshold for a single-value check (which evaluates just total acceleration), with all thresholds updating dynamically with each swing completion. (Other embodiments may use constant values for these thresholds, but that approach may trigger false-positives if the constant thresholds are relatively low, or may fail to detect disruptions if the constant thresholds are relatively high. By contrast, having the thresholds update dynamically allows them to be tailored to any level of accelerometer activity for different users, equipment, etc.) For this embodiment, the moderate range-of-motion threshold is calculated as the greatest range-of-motion of the last four completed swings plus 10.0°, the moderate total acceleration threshold is calculated as the last completed swing's highest total acceleration value plus 3.0 g-forces, and the severe total acceleration threshold is calculated as the last completed swing's highest total acceleration value plus 5.0 g-forces with a maximum value of 8.0 g-forces (though other embodiments may use alternative threshold calculations).

In step 2440, this embodiment checks whether the sign multipliers should be updated based on potential changes in the sensor assembly's roll. (In this embodiment, the sign multipliers are values of 1 or −1 by which the gyroscopic values on the non-proximal axes may be multiplied before being added together to calculate the interpreted gyroscopic value.) For this embodiment, the sign multipliers are handled differently depending on the sensor assembly's pitch. Specifically:
If the pitch is angled (in which case the roll is known right away), both non-proximal axes have sign multipliers based on the orientations of their local positive directions of gyroscopic rotation as depicted in FIG. 21. With that basis, the sign multiplier for the perpendicular axis is negative if the roll is right of center (and vice versa), and the sign multiplier for the transverse axis is −1 if the roll is front of center (and vice versa). The result of this is, when adding the products of each non-proximal axis gyroscopic value times its respective sign multiplier, a positive sum indicates upward rotation, while a negative sum indicates downward rotation.
If the pitch is upright (in which case the roll estimation may be awaiting motion analysis), the gyroscopic value on the primary axis is used directly (i.e. without a sign multiplier) while the gyroscopic value on the secondary axis is multiplied by the primary-secondary sign multiplier established by the second calibration method (as described for step 2732). The result of this is, when adding the primary axis direct gyroscopic value with product of the secondary axis gyroscopic value times the primary-secondary sign multiplier, a positive sum indicates rotation in a direction that causes positive gyroscopic values on the primary axis, while a negative sum indicates rotation in a direction that causes negative gyroscopic values on the primary axis. (These directions may be identified as forward and backward using logic from the first calibration method, as described for step 2462).

In step 2450, this embodiment checks for short-swings as a means of shake detection for repetitive shaking motion (e.g. the user bobbing a knee up and down) that may have made it past the range-of-motion requirement for single swings. (If detected, this may trigger a software response for the user to see, though it may be preferable to make this response less intrusive than that for a “sensor disruption” as described for step 2333). This check evaluates whether a short-swing count threshold has been met by multiple recent swing ranges-of-motion (as cached in step 2410) falling under the short-swing range-of-motion threshold (as set in step 2420). For this embodiment, the short-swing count threshold is 4 swings in a row (though alternate embodiments may use other thresholds). (The number of ranges checked may vary by context and developer preference, but it is generally preferred to check multiple, consecutive ranges to avoid oversensitivity to single swings with lower-than-expected ranges.)

In decision 2460, this embodiment checks whether the sensor assembly's pitch is upright (this is estimated by step 2511 using logic from the first calibration method).

The remaining steps in FIG. 24 (2461-2490) execute if the pitch is upright (in which case the sensor assembly's roll and each swing's direction are based on motion analysis rather than accelerometer response to the pull of gravity).

In step 2461, this embodiment determines the new swing's estimated direction. (This step also caches the previous swing's estimated direction, which may be used for the new swing direction determination and/or for estimating the sensor assembly's roll about the proximal axis.) If the roll estimation has already been finalized and a threshold count of alternating swing directions (e.g. 4 in a row, though alternate embodiments may use other thresholds) has been met in the current activity session, the new swing's direction may be determined by simply alternating the direction from the previous swing (e.g. if the embodiment is confident that the previous swing was backward, the new swing is assumed to be forward without need for motion data analysis). However, if either the roll estimation is not yet finalized or the alternating direction threshold count has not yet been met, then step 2461 uses logic from the first calibration method to analyze the last two swings' highest vertical accelerations. The logic for forward/backward estimation is described below.

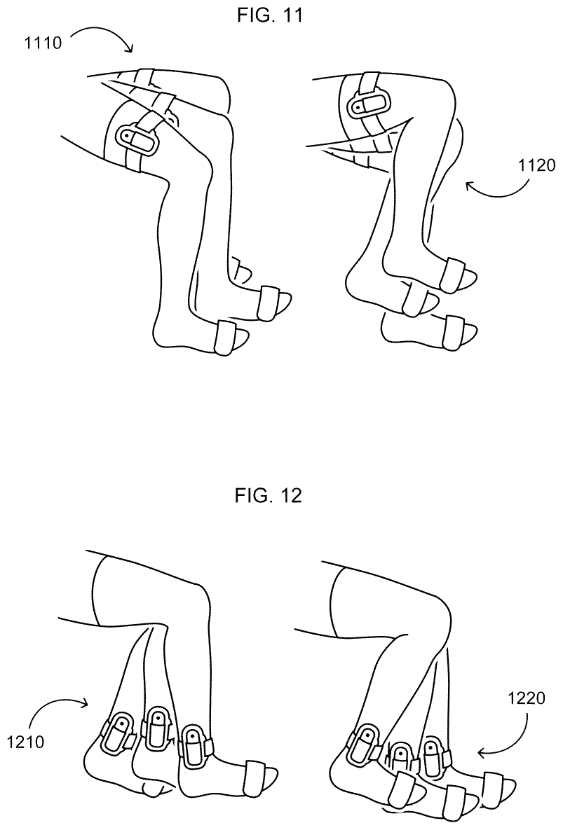

Given the sensor assembly at an upright pitch, vertical acceleration is expected to be highest when the sensor assembly's linear motion changes from downward to upward. This happens at the lowest point in the pedaling motion, which occurs during the back-swing phase (as depicted by FIG. 12 at 1220). Therefore, if the latest completed swing's highest vertical acceleration exceeds that of the swing before it, then the latest completed swing's direction is estimated to be backward, and the newly started swing's direction is estimated to be forward. (It is notable that accelerometer sensitivity to bumps and shakes may introduce a level of uncertainty into this estimation. This is why the embodiment seeks multiple estimates in an alternating forward/backward pattern rather than using a single estimation.)

In decision 2470, this embodiment checks if the new swing's direction estimate used accelerometer analysis from previous swings (as opposed to a simple alternation from the last swing's direction). To recap, accelerometer analysis is used if either the roll is not yet finalized or the alternating direction count has not reached a threshold (as described in step 2462). If the check returns true, then step 2471 executes next; otherwise decision 2480 executes next.

In step 2471, this embodiment checks if the latest estimated swing direction differs from the previous swing's direction estimate. If so, the count of alternating directions increments by one; if not, the count of alternating directions resets to zero. This effectively ensures motion analysis continues for swing direction and roll estimation until multiple swings in a row have yielded results consistent with an expected pattern.

In decision 2480, this embodiment checks whether the sensor assembly's roll is still pending further estimation. Specifically, this evaluates whether the variable for sensor assembly roll is set to a non-finalized value (e.g. an enumerator may be set to “Awaiting Motion” or “Analyzing” before being finalized by the process depicted in FIG. 26, which is described further below). If the check returns true, then step 2481 executes next; otherwise step 2490 executes next.

In step 2481, this embodiment uses logic from the first calibration method to estimate the sensor assembly's roll based on analysis of motion data from the previous swing. The logic for this is expanded in FIG. 26 and described in detail further below.

In step 2490, this embodiment resets preparation for upright direction estimation. (This is contingent on further motion analysis still being used for direction estimates, using the same condition checks as decision 2481.) Specifically, this caches the highest vertical acceleration for the latest completed swing as a variable saved for the previous swing, and resets the corresponding value monitored for the current swing to 0 (resetting it to be overwritten as the new swing progresses).

### First Reading and Intervals—Orientation Estimation

At regular intervals (e.g. 10 times per second, though alternate embodiments may use other intervals) this embodiment executes the logic depicted in FIG. 25. This uses logic from the first calibration method for estimating whether the sensor assembly's pitch is upright or angled, and estimating the sensor assembly's roll about the proximal axis if the pitch is determined to be angled. This logic also includes instructions for how to respond if estimations of the sensor assembly's pitch waver between upright and angled.

In decision 2510, this embodiment checks if the first swing is still pending. This check returns true if the first swing since a “stopped” status has not yet completed (e.g. if the user is at rest, or is so early in a pedaling motion that the first swing is still underway). This is achieved by checking if the pedaling confidence is 0 (as the value defaults to 0 while the status is stopped, and rises to 1 upon completion of the first swing). If the check returns true, then step 2511 executes next; otherwise decision 2520 executes next.

In step 2511 (executing if the check in decision 2510 returns true), this embodiment estimates the sensor assembly's pitch as upright or angled. This estimation is determined by analyzing accelerometer response to the pull of gravity on the sensor assembly's axes. Given this embodiment's guidelines for orientations of the sensor assembly's proximal, perpendicular, and transverse axes, an upright pitch is indicated by strong accelerometer response to the pull of gravity on the proximal axis, and therefore weak accelerometer response to gravity on the non-proximal axes. As described in step 2220, this embodiment smooths the non-proximal axes' accelerometer values by averaging each over time, effectively isolating the effect of gravity. Therefore, this embodiment estimates the sensor assembly's pitch using the sum of the absolute values of the averaged accelerometer values on the non-proximal axes (as opposed to a single, less-smoothed value for the proximal axis, which may be more oversensitive to bumps, shakes, etc.). If the sum of the absolute values of the averaged accelerometer values on the non-proximal axes is under a threshold, then the pitch is determined to be upright (e.g. for this embodiment, a threshold of 0.8 g-force is effective for consistently identifying thigh-mounted pitches as angled, and ankle-mounted pitches as upright, though other embodiments may use alternative thresholds).

(Alternative embodiments may estimate pitch as a matter of extent or degree, rather than a binary determination of “upright” or “angled.” Such estimation could still use the same principles applied in this embodiment, e.g. by analyzing a ratio of gravity's effect on the proximal axis compared to the non-proximal axes. For this embodiment, however, a binary determination is sufficient for pitch-based evaluations.)

Since step 2511 is conditional on decision 2510, this embodiment's window for changing the sensor assembly's pitch estimation is limited to before or during the first swing of a pedaling session. This maintains some level of flexibility for pitch estimation to recalculate while the user is at rest, but prevents the pitch estimation from wavering mid-swing once the user is actively pedaling. In addition to estimating the sensor assembly's current pitch, step 2511 also caches the value for the sensor assembly's prior pitch (which may be checked in decision 2530 or 2540 to detect if the pitch has changed).

In decision 2520, this embodiment checks if the sensor assembly's pitch has been estimated as upright. (This may have just been set by step 2511 if a pedaling session's first swing is not yet complete, or it may be “locked in” for a pedaling session after completion of the first swing.) If the pitch is upright, then the embodiment proceeds to decision 2530; otherwise, the embodiment proceeds to decision 2540.

In decision 2530 (executing if the sensor assembly's current pitch is upright), this embodiment checks if the sensor assembly's prior pitch was upright. A return of true here indicates the upright pitch estimation is consistent with the previous check, and the remaining steps in FIG. 25 may be bypassed this time-interval check. A return of false here indicates the pitch estimation has just changed from angled to upright, and step 2531 executes in response to this.

In step 2531 (executing if the sensor assembly's pitch just changed from angled to upright), this embodiment resumes the roll estimation that was cached the last time the pitch was estimated to be upright. If that cached roll was not yet finalized (e.g. “Awaiting Motion” or “Analyzing . . . ”), then the upright roll estimation is still subject to the logic from the first calibration method depicted in FIG. 26 and described in detail further below. However, if the roll was already finalized before the pitch briefly changed to angled and then returned to upright, this step allows resuming that finalized pitch without running the estimation process over again.

In decision 2540 (executing if the sensor assembly's current pitch is angled), this embodiment checks if the sensor assembly's prior pitch was upright. A return of true here indicates the pitch estimation has just changed from upright to angled, and step 2541 executes in response to this. A return of false here indicates the sensor assembly's angled pitch estimation is consistent with the previous check, and step 2542 executes in response to this.

In step 2541 (executing if the sensor assembly's pitch just changed from upright to angled), this embodiment caches the last roll estimation value from when the pitch was upright. (If the sensor assembly later resumes an upright pitch, the roll estimation will resume this value in step 2531 during another loop of the FIG. 25 logic.)

In step 2542 (executing if the sensor assembly's pitch is angled, regardless of prior status), this embodiment caches the roll type (i.e. orthogonal or diagonal) and primary and secondary designations for the non-proximal axes. (Between the axes that may measure swing rotation, those being the non-proximal axes for this embodiment, the primary axis is the one that should have more gyroscopic activity when tracking the intended activity). Both determinations compare accelerometer response to gravity on the sensor assembly's non-proximal axes to estimate how gravity is pulling on the sensor assembly in its current orientation.

For step 2542′s roll type determination, the principle considered is that (given an angled pitch) an orthogonal roll results in gravity pulling significantly more on one non-proximal axis than the other, while a diagonal roll results in gravity pulling noticeably on both non-proximal axes. Therefore, this embodiment determines the roll type by comparing the ratio of the absolute values of the averaged accelerometer values on the non-proximal axes; if one average's absolute value exceeds the other by a given extent, then the roll type is determined to be orthogonal, and otherwise the roll type is determined to be diagonal. For this embodiment, this check specifically evaluates whether either non-proximal axis has an absolute value of averaged accelerometer readings greater than 250% the other's, determining the roll type is orthogonal if so and diagonal if not (though other embodiments may use alternative thresholds).

For step 2542′s primary and secondary axis identifications, the first calibration method's accelerometer-based approach differs from how the axis identifications are handled in the second calibration method's sample. The accelerometer-based approach operates on the principle that, given an angled pitch, the intended activity will result in more gyroscopic rotation on the axis with less accelerometer response to the pull of gravity (as can be visualized via the axis indicators in FIGS. 18 and 19). Specifically, this step identifies the primary and secondary axes by checking which non-proximal axis has the accelerometer average with the higher absolute value: if gravity is pulling more on the perpendicular axis, then the transverse axis is the primary, and vice versa.

In decision tree 2550 (also executing if the sensor assembly's pitch is angled), this embodiment determines the sensor assembly's roll based on accelerometer readings and the values cached in step 2542. The checks herein evaluate whether the roll is orthogonal (as determined in 2542), centered (indicated by the transverse axis being the primary axis), front-facing (indicated by the accelerometer average for the perpendicular axis being positive), and/or left-facing (indicated by the smoothed accelerometer average for the transverse axis value being positive). The results of those four evaluations yield eight possible outcomes for the sensor assembly's roll:
Orthogonal, centered, front-facing: Front
Orthogonal, centered, back-facing: Back
Orthogonal, not centered, left-facing: Left
Orthogonal, not centered, right-facing: Right
Diagonal, front-facing, left-facing: Front-left
Diagonal, front-facing, right-facing: Front-right
Diagonal, back-facing, left-facing: Back-left
Diagonal, back-facing, right-facing: Back-right

In decision 2560 (executing after roll estimation), this embodiment checks if the sign multipliers have been set. (For more details on the role of sign multipliers for setting the interpreted gyroscopic value, see the description for step 2430.) For this embodiment, the sign multipliers begin at 0 by default; if they are still set to that value, then step 2561 executes next to update their values. (If the sign multipliers have already been set to non-zero values, they are left alone until the “Each New Swing” logic depicted in FIG. 24 and described above-this prevents the sign multipliers from changing mid-swing, which could cause erratic interpretation of the sensor assembly's motion.)

In step 2561 (executing if the sign multipliers have not yet been changed from 0), this embodiment sets the sign multiplier values to their initial non-zero values of 1 or −1. To recap, for this embodiment, the perpendicular axis sign multiplier should be −1 if the sensor assembly is right-facing (and vice versa), and the transverse axis sign multiplier should be −1 if the sensor assembly is front-facing (and vice versa). This step assigns those values using the same evaluations from decision tree 2550.

If the sensor assembly's pitch is angled, the logic described above can estimate the roll with just a single reading's accelerometer values. However, if the pitch is upright, then roll estimation is handled by a multi-stage process that executes each time a new swing begins; this process is described below.

### Each New Swing−Roll Estimation if Pitch Is Upright

If the sensor assembly's pitch is upright, then this embodiment estimates its roll using logic from the first calibration method to analyze data from completed swings. To recap for context, each time a new swing begins (as checked for in step 2331), the new swing initiation logic (depicted in FIG. 24) includes checks for whether the sensor assembly's pitch is upright (decision 2460) and whether further roll estimation is called for (decision 2480). If both these checks return true, then step 2481 executes, which is expanded in FIG. 26 and described in detail here.

In decision 2610, this embodiment checks if the sensor assembly's roll type (i.e. orthogonal or diagonal) is already finalized. Specifically, this checks a count of previous swing completions that have had consistent, consecutive results using the evaluation logic described below; if this count has met a given threshold (e.g. 3 consistent results in a row, though alternate embodiments may use other thresholds), the result is considered confident enough to finalize.

In step 2611, this embodiment estimates the sensor assembly's roll type (i.e. orthogonal or diagonal). Unlike the logic for an angled pitch, at an upright pitch the accelerometer values on the non-proximal axes are not strong indicators of roll (since the pull of gravity is primarily on the proximal axis). Therefore, this embodiment analyzes sums of the previous swing's total gyroscopic motion on the perpendicular and transverse axes (these sums having been gathered as part of the mid-swing value updates described for step 3061 below). The principle here is that an orthogonal roll yields significantly stronger gyroscopic readings on one axis than the other (as depicted in FIGS. 18 and 19), while a diagonal roll yields more of a mix of gyroscopic readings on both axes (as depicted in FIG. 20). Therefore, this embodiment determines the roll type by comparing the ratio of the absolute values of the previous swing's gyroscopic sums; if one sum exceeds the other by a given extent, then the roll type is determined to be orthogonal, and otherwise the roll type is determined to be diagonal. Specifically, this embodiment reviews the previous swing and evaluates if either gyroscopic sum exceeds 200% the other gyroscopic sum, determining the roll type is orthogonal if so and diagonal if not (though other embodiments may use alternative thresholds). Before setting this latest roll type estimation, this step also caches the estimation's previous value for comparison.

In decision 2620, this embodiment checks if the new roll type estimate matches the previous estimate. (This check also allows for a true return if there was no previous estimate, i.e. if the new value is the first estimation since a reset.)

In step 2621 (executing if the new roll type estimate matches the previous one), this embodiment increments the count of consistent roll type estimates by 1 (i.e. bringing the estimation system one step closer to finalization).

In step 2622 (executing if the new roll type estimate does not match the previous one), this embodiment resets the count of consistent roll type estimates to 0 (i.e. resetting the estimation system to start a new count of consistent-results-in-a-row before finalization).

In step 2630, this embodiment caches values from the completed swing's analysis. These values comprise variables (e.g. true/false booleans) for quick-access checks as to whether:
The swing's direction was forward (as determined by step 2461)
The swing's gyroscopic sum for the perpendicular axis was positive
The swing's gyroscopic sum for the transverse axis was positive
The gyroscopic sums share the same positive or negative sign

Decision 2640 marks the start of this embodiment's logic tree for estimating upright sensor assembly roll based on the gyroscopic sums and the values cached in 2630. The arrangement of evaluations here is similar to FIG. 25's decision tree at 2550, but due to the upright pitch, the evaluation results here are determined by analyzing swing data rather than accelerometer values. (To recap, 2550 determined pitch by comparing the pull of gravity on the non-proximal axes, while an upright pitch limits gravity's effect primarily to the proximal axis.)

Branch 2641 marks a continuation of this embodiment's logic for estimating upright roll if the roll type is orthogonal (to recap, this means one gyroscopic sum's absolute value is more than double the other's).

(For context on how aspects of the sensor assembly's roll are derived from swing directions and the positive or negative signs of gyroscopic rotations, see the chart of gyroscopic rotation sign directions by roll depicted in FIG. 21.)

Given an orthogonal roll, the embodiment determines if the sensor assembly is centered (i.e. front/back roll, as opposed to left/right) by comparing the sums' absolute values—for this embodiment's sensor assembly, a higher absolute value on the transverse axis indicates centered placement, while a higher absolute value on the perpendicular axis indicates sideways placement. Given a centered result the roll is determined to be front-facing if either the swing was forward and the transverse axis gyroscopic sum was negative, or the swing was backward and the transverse axis sum was positive; otherwise it is determined to be back-facing. Given a sideways result, the roll is determined to be left-facing if either the swing was forward and the perpendicular axis gyroscopic sum was positive, or the swing was backward and the perpendicular axis gyroscopic sum was negative; otherwise it is determined to be right-facing.

Branch 2642 marks a continuation of this embodiment's logic for estimating upright roll if the type is diagonal (again, meaning there was significant gyroscopic rotation on both non-proximal axes). Given a diagonal roll, the embodiment uses the same conditions described for branch 2641 to determine if the sensor assembly is generally front-facing (indicated by a forward swing with a negative transverse axis gyroscopic sum, or a backward swing with positive transverse axis gyroscopic sum) and/or left-facing (indicated by a forward swing with a positive perpendicular axis gyroscopic sum, or a backward swing with a negative perpendicular axis gyroscopic sum). The combination of results for these front/back and left/right checks ultimately determines which diagonal roll (front-left, front-right, back-left, or back-right) is estimated.

In step 2650, this embodiment resets values for the next round of upright roll estimation. This entails setting the gyroscopic sums for the non-proximal axes back to 0, to then be updated as the new swing progresses.

### Each Reading—Updating Motion Sample

As this embodiment executes FIG. 23's logic each reading, step 2310 gathers data for the second calibration method's sample (if called for). This sample is conducted when the user starts pedaling while the sensor assembly's pitch is upright and its roll estimation is not yet finalized. In this embodiment, the sample serves to identify the primary axis, the secondary axis, and the primary-secondary sign multiplier; this data can then be analyzed to start tracking swings, even if the upright roll has not yet been estimated. The logic for this is expanded in FIG. 27 and described in detail here.

Hypothetically, embodiments using the second calibration method could identify any axis as primary, secondary, or tertiary. However, this embodiment does not expect significant rotation about the proximal axis during performance of the intended activity. Therefore, this embodiment's use of the second calibration method only samples rotation on the two non-proximal axes to determine which is primary and which is secondary.

In decision 2710, this embodiment checks if the sample should proceed this reading. This check returns true if the sensor assembly is upright and the sample results are not yet finalized. (Information on sample finalization is provided in step 2750.) If this return is false (indicating the sample is not called for), the remaining steps in FIG. 27 may be bypassed this reading. If the return is true, step 2711 executes next.

In step 2711, this embodiment updates the sample's data. This adds the current reading's absolute values of gyroscopic rotation on the perpendicular and transverse axes to respective “perpendicular sampled rotation” and “transverse sampled rotation” variables that cache the cumulative amount of gyroscopic rotation on each axis during the sample. (Absolute values are preferred here rather than raw values so that, if the sample spans multiple back-and-forth swings, the gyroscopic readings' alternating positive and negative values do not cancel each other out.) This step also multiplies the signs of the perpendicular and transverse gyroscopic values together and caches the product in an array for later use in setting the primary-secondary sign multiplier. (The cached product is 1 if the signs are the same, or −1 if the signs are different.)

In decision 2720, this embodiment evaluates if the sample is ready for its results thus far to be checked. A series of thresholds here may be preferable so the sample results can be checked multiple times before being finalized—if the sample's results were only checked once, then requiring a high amount of data may introduce lag before allowing swings to be tracked (i.e. delaying the status transition from “stopped” to “moving”), while requiring a low amount of data may reduce confidence in the sample's results (i.e. increasing the risk of an incorrect primary axis identification). Therefore, decision 2720 may return true at a series of thresholds as the sample proceeds—the first threshold's preliminary results allow the embodiment to quickly begin tracking swings with the sensor assembly upright, and the subsequent thresholds allow the embodiment to revise the sample's outcome if additional data indicates a change in results (e.g. a different primary axis than the one previously identified). This embodiment's decision 2720 returns true each time the sum of the perpendicular sampled rotation and transverse sampled rotation first crosses any of the thresholds 10°, 30°, 50°, and 70° (though alternative embodiments may use other thresholds). If this check's return is false, the remaining steps in FIG. 27 may be bypassed this reading, and the motion sample will continue gathering data in the next reading. If the return is true (i.e. each time the total sum of sampled values crosses a new threshold), decision 2730 executes next.

In decision 2730, this embodiment evaluates if the sample's data gathered thus far indicates a change in results (either compared to results of previous checks, or because the sample is checking results for the first time). This step entails comparing the perpendicular sampled rotation and transverse sampled rotation, with the greater value indicating the primary axis of rotation, and analyzing the array of sign products cached by step 2711 to determine the primary-secondary sign multiplier. The sign multiplier is set to 1 if the majority of cached products are positive (indicating most of the sampled readings had the same sign on both axes), or −1 if the majority of cached products are negative (indicating most of the sampled readings had different signs on the non-proximal axes). Decision 2730 returns true if the primary axis has not yet been identified (e.g. if the sample is checking its results for the first time), if the newly identified primary axis differs from the one previously identified (e.g. if an initial wobble in the user's leg started rotating the sensor assembly primarily on one axis, but the sensor assembly's ongoing rotation is primarily on the other), or if the newly determined sign multiplier differs from the one previously determined (i.e. if the sample indicates a different relationship between the axes' local positive directions than previously inferred). If this return is true, step 2731 executes next; otherwise step 2750 executes next.

In step 2731 (executing if the sample has identified a change in results), this embodiment caches which axis has been identified as the primary axis, as determined by the results check in decision 2730, with the other non-proximal axis then understood to be the secondary axis.

In step 2732 (also executing if the sample has identified a change in results), this embodiment caches the primary-secondary sign multiplier, as determined by the results check in decision 2730. (For more on how this value is used, see the description for step 2811.)

In decision 2740 (also executing if the sample has identified a change in results), this embodiment checks if a primary axis had already been set by an earlier sample check. This is based on a count of times that the sample results have been checked, with any value over 0 resulting in a return of true. If the return is true, step 2741 executes next; otherwise step 2750 executes next.

In step 2741 (executing if a primary axis had already been set before the sample identified a change in results), this embodiment resets values related to swing status and progression. These values include the swing status (primary positive or primary negative), the pedaling confidence, and the current swing duration, mid-swing shake count, and highest and lowest angles for range-of-motion estimation. (When the sample results change, resetting these values provides a “clean slate” for the following swing-tracking to be based on shared, consistent sample results.)

In step 2750 (executing each time the sample checks its results, regardless of whether or not this changes the primary axis), this embodiment increments the count of times that its results have been checked. (Once this count reaches the number of thresholds described in decision 2710, the embodiment considers the motion sample complete.)

### Each Reading—Interpreting Gyroscopic Values

As this embodiment proceeds through FIG. 23's logic each reading, step 2320 interprets gyroscopic data from the sensor assembly to set the interpreted gyroscopic value. This process determines the reading's degrees and direction swung, which this embodiment also used as a basis for checking the sensor assembly's status as “moving” or “stopped.” The logic for this process is expanded in FIG. 28 and described in detail here.

In decision 2810, this embodiment checks if the sensor assembly's pitch is angled. (For more on how this is determined, see the description for step 2520.) If the pitch is angled, then step 2811 executes next; otherwise, step 2812 executes next.

In step 2811 (executing if the pitch is angled), this embodiment uses the first gyroscopic interpretation method to set the interpreted gyroscopic value given an angled pitch. This entails reading the sensor assembly's gyroscopic values for the perpendicular and transverse axes, multiplying each by their respective sign multiplier, and adding the resulting values together for an interpreted sum. (To recap, sign multipliers for an angled pitch are initially set in step 2561, and they may be updated with each new swing in step 2430.)

As a result, step 2811 provides an interpreted gyroscopic value for angled sensor assembly motion in which the absolute value indicates degrees of rotation, a positive value indicates the rotation is upward, and a negative sign indicates the rotation is downward.

In step 2812 (executing if the pitch is upright), this embodiment uses the second gyroscopic interpretation method to set the interpreted gyroscopic value given an upright pitch. Since the sensor assembly's roll may not yet be known in this context (pending consistent estimations by FIG. 26′s decision tree starting at 2640), this step interprets the motion data using the second calibration method's primary and secondary axis identifications from step 2731 and the primary-secondary sign multiplier from step 2732. This interpretation entails adding the primary axis gyroscopic value to the product of the secondary axis gyroscopic value times the primary-secondary sign multiplier for an interpreted sum.

As a result, step 2812 provides an interpreted gyroscopic value for upright sensor assembly motion in which the absolute value indicates degrees of rotation, a positive value indicates the rotation is in a direction that the primary axis detects as positive, and a negative sign indicates the rotation is in a direction that the primary axis detects as negative. (With sufficient data available, these directions may be deemed “forward” or “backward” by step 2461.)

In step 2820, this embodiment caches the reading's interpreted gyroscopic value from either step 2811 or 2812 (depending on the return from decision 2810). To increase the result's smoothness (e.g. to reduce sensitivity to shaking in the user's leg when first starting to pedal against resistance), this embodiment adds the current reading's value in an array of recent values and averages the array's recent contents (e.g. from the last tenth of a second). Given the interpretation logic from step 2811 or 2812 and the smoothing utilized here, the resulting interpreted gyroscopic value is cached as a smoothed estimate for how many degrees the sensor assembly has swung through the intended activity in the time since the previous reading.

With the interpreted gyroscopic value set, steps 2830-2842 offer a means of detecting motion lateral to the intended activity (e.g. from swaying the knee from side to side instead of pedaling). This embodiment uses this detection when motion is first detected while the sensor assembly's status is “stopped” (i.e. if the user has been at rest, the embodiment is designed to trigger a desired response if the user begins pedaling, but not if the user begins swaying a knee).

Specifically, this embodiment makes the transition from “moving” to “stopped” conditional on a brief period of time (e.g. 0.1 seconds, though alternative embodiments may use other thresholds) in which the sensor assembly's motion is consistently determined to be non-lateral (i.e. in the directions expected for the intended activity). This embodiment checks for this condition being met by a count of consecutive non-lateral readings, deemed a “non-lateral reading count.”

In decision 2830, this embodiment checks if a non-lateral reading count is being monitored. This returns true if the sensor assembly's status is “stopped,” the interpreted gyroscopic value is over the threshold checked for the “moving” status (as set by step 2430), and the count of non-lateral readings so far is lower than the threshold for entering the “moving” status (e.g. the number of readings in 0.1 seconds, though alternative embodiments may use other thresholds). If the return is true, then decision 2840 executes next; otherwise step 2850 executes next.

In decision 2840 (executing if the non-lateral reading count is being monitored), this embodiment checks if the detected motion is lateral. The means of checking this depends on whether rotation from the intended activity is expected to be single-axis or multi-axis. (Here the term “single-axis” is used in a general sense, referring to rotation primarily detected on a single axis, not exclusively detected on a single axis.) This may be determined through accelerometer readings if the sensor assembly's pitch is angled, or through the ratio of sampled primary and secondary rotation values if the pitch is upright. These determinations are similar in principle to the evaluations of whether the sensor assembly's roll type is orthogonal or diagonal (as determined through accelerometer readings in step 2542, or through a primary/secondary comparison in step 2611). In short, if a determination suggests the roll type is orthogonal, then it also suggests rotation from the intended activity is expected to be single-axis; if a determination suggests the roll type is diagonal, then it also suggests rotation from the intended activity is expected to be multi-axis.

If rotation from the intended activity is expected to be single-axis, then the primary axis should have significantly more gyroscopic rotation than the secondary axis; therefore lateral motion may be indicated by the secondary axis absolute value of gyroscopic rotation over-exceeding that of the primary axis. Specifically, when expecting single-axis rotation, this embodiment detects motion as lateral if the absolute value of secondary axis gyroscopic rotation exceeds 150% the absolute value of primary axis rotation (though alternative embodiments may use other thresholds).

If rotation from the intended activity is expected to be multi-axis, then instead of comparing amounts of rotation on the axes, lateral motion may be detected by comparing the positive or negative signs of the values added when calculating the interpreted gyroscopic value. (To recap, these values are the proximal and transverse axes' gyroscopic values each times their respective sign multipliers if the pitch is angled, or the direct primary axis gyroscopic value and the product of the secondary axis gyroscopic value times the primary-secondary sign multiplier if the pitch is upright.) During performance of the intended activity, the addend values for the interpreted sum should have the same positive or negative sign, so lateral motion may be detected if those values' signs do not match.

Even if logic above detects lateral motion, the detection may be “excused” if the sensor assembly has an upright pitch and the second calibration method' sample is relatively early in its analysis. Therefore, a return of true for decision 2840 is also conditional on the sensor assembly's pitch being angled, or the count of motion sample result checks being at or over a threshold (e.g. at least 2 checks for this embodiment, though alternative embodiments may use other thresholds).

If the return for decision 2840 is true (indicating detection of lateral motion), then step 2841 executes next; otherwise step 2842 executes next.

In step 2841 (executing if lateral motion was detected), this embodiment resets the count of non-lateral readings to zero. Effectively, if the sensor assembly's status is not yet set to “moving,” a reading of lateral motion results in this embodiment restarting its count of non-lateral readings checked before entering the “moving” status.

In step 2842 (executing if lateral motion was not detected), this embodiment increments the count of non-lateral readings by one. Effectively, if the sensor assembly's status is still “stopped,” a reading of non-lateral motion brings the system a step closer to readiness for the “moving” status.

In decision 2850, this embodiment checks if the interpreted gyroscopic value's absolute value is equal to or greater than the minimum threshold for the “moving” status (as set in step 2430). If the return is true, step 2851 executes next; otherwise, step 2852 executes next.

In step 2851 (executing if the interpreted gyroscopic value's absolute value is equal to or greater than the minimum threshold), this embodiment checks if the sensor assembly's status is currently “stopped” and if the count of non-lateral readings is at or over the threshold for entering the “moving” status. If so, this step sets the sensor assembly's status to “moving.” This response also checks a timer value for how long the sensor assembly has gone without moving; if that value is greater than zero, this step resets it to zero when setting the “moving” status.

In step 2862 (executing if the interpreted gyroscopic value's absolute value is under the minimum threshold), this embodiment checks if the sensor assembly's status is currently “moving,” and if so, updates a timer value for how long the sensor assembly's motion has stayed under the “moving” threshold. If that value exceeds the time threshold for entering the “stopped” status (as set in step 2430), then this step also changes the sensor assembly's status from “moving” to “stopped.” This status change also results in a variety of values being set for the “stopped” status, as listed in step 2210.

### Each Reading—Checking for Swing or Mid-Swing Shake

As this embodiment proceeds through FIG. 23's logic each reading, step 2331 checks for whether a new swing is detected. The logic for this is expanded in FIG. 29 and described in detail here. As it pertains to direction changes, this logic also includes checks for detecting individual, mid-swing shakes.

In decision 2910, this embodiment first checks if calibration is ready to track swings at all. This check returns true if the sensor assembly's pitch is angled (as set by step 2511), in which case the first calibration method has ascertained the sensor assembly's orientation, or if the pitch is upright and the second calibration has checked its sample results at least once (as counted by step 2750). If the return is true, step 2911 executes next; otherwise, the remaining steps in FIG. 29 may be bypassed this reading.

In decision 2920, this embodiment checks if the system is ready to initiate a new swing. A return of true here is usually conditional on the swing's duration being over a minimum value (e.g. 0.15 seconds, effectively preventing a cadence value higher than 200 RPM) and the swing's range-of-motion (from step 3030) being over the dynamic range-of-motion requirement (from step 2430). However, this check may also return true if the pedaling confidence is 0 or 1 (effectively waiving the duration and range-of-motion requirements for the initial motion when the user starts pedaling). If the return is true, decision 2930 executes next; otherwise, decision 2960 executes next.

In decision 2930 (executing if the system is ready to initiate a new swing), this embodiment checks if the sensor assembly's pitch is upright (as set by step 2511). If the return is true, decision 2940 executes next; otherwise decision 2950 executes next.

Depending on the sensor assembly's pitch, this embodiment may or may not be ready to identify the direction of a new swing as it begins. Since an upright pitch (as pertaining to steps 2940-2943) uses motion analysis to determine swing direction (as described in step 2461), newly detected swings for an upright pitch may simply be deemed “primary positive” and “primary negative” (referring to whether the swing results in positive or negative gyroscopic values on the primary axis, rather than the swing's direction relative to the user). By contrast, an angled pitch (as pertaining to steps 2950-2953) facilitates roll and direction estimation without ongoing motion analysis, and therefore newly detected swings for an angled pitch may be identified as “up” and “down” (referring to the sensor assembly's direction of motion).

In decision 2940 (executing if the pitch is upright while the system is ready for a new swing), this embodiment checks if a new primary positive swing is detected. This check returns true if the current swing type is “none” or primary negative and the interpreted gyroscopic value (from step 2820) is positive. If the return is true, step 2941 executes next; otherwise decision 2942 executes next.

In step 2941 (executing if 2940 detected a new primary positive swing), this embodiment initiates tracking the new primary positive swing. This executes the new-swing logic depicted in FIG. 24 and sets the current swing type as primary positive.

In decision 2942 (executing if 2940 did not detect a new primary positive swing), this embodiment checks if a new primary negative swing is detected. This check returns true if the current swing type is “none” or primary positive and the interpreted gyroscopic value (from step 2820) is negative. If the return is true, step 2943 executes next; otherwise, the remaining steps in FIG. 29 may be bypassed this reading.

In step 2943 (executing if 2942 detected a new primary negative swing), this embodiment initiates tracking the new primary negative swing. This executes the new-swing logic depicted in FIG. 24 and sets the current swing type as primary negative.

In decision 2950 (executing if the pitch is angled while the system is ready for a new swing), this embodiment checks if a new up-swing is detected. This check returns true if the current swing type is “none” or down-swing and the interpreted gyroscopic value (from step 2820) is positive. If the return is true, step 2951 executes next; otherwise decision 2952 executes next.

In step 2951 (executing if 2950 detected a new up-swing), this embodiment initiates tracking the new up-swing. This executes the new-swing logic depicted in FIG. 24 and sets the current swing type as up-swing.

In decision 2952 (executing if 2950 did not detect a new up-swing), this embodiment checks if a new down-swing is detected. This check returns true if the current swing type is “none” or up-swing and the interpreted gyroscopic value (from step 2820) is negative. If the return is true, step 2953 executes next; otherwise, the remaining steps in FIG. 29 may be bypassed this reading.

In step 2953 (executing if 2952 detected a new down-swing), this embodiment initiates tracking the new down-swing. This executes the new-swing logic depicted in FIG. 24 and sets the current swing type as down-swing.

In steps 2960-2981 (executing if decision 2920 determined the system is not ready to initiate a new swing), this embodiment checks for and counts “mid-swing shakes” (changes of rotation direction before a swing's minimum range-of-motion requirement has been met, which do not result in changing to a new swing, with the minimum range-of-motion requirement having been set in step 2430). Depending on developer preference, these may be used to trigger software response (e.g. displaying a message if the mid-swing shake count exceeds a given threshold), or simply ignored (i.e. allowing the activity tracking to proceed, assuming the swing eventually meets the minimum range-of-motion requirement).

In decision 2960, this embodiment checks if a “shake-ready” variable is set true. (This serves as a condition for counting mid-swing shakes; the embodiment may have it set true by default, then set it false when a shake is counted, then true again when a swing resumes its expected direction of motion.) If the return is true, then decision 2970 executes next; otherwise, decision 2980 executes next.

In decision 2970 (executing if the shake-ready variable is set true), this embodiment checks if the interpreted gyroscopic sum indicates the sensor assembly's gyroscopic motion has changed direction. This check returns true if the conditions are met for any of the evaluations from decisions 2940, 2942, 2950, or 2952 (i.e. meaning it appears the sensor assembly has switched from one swing to another, but here in the context that the swing's range-of-motion is under the minimum requirement). If the return is true, then step 2971 executes next; otherwise decision 2980 executes next.

In step 2971, this embodiment updates the shake count for the current swing. This step also sets the shake-ready variable false (this prevents a single change of direction that spans multiple readings from being counted as multiple shakes).

In decision 2980 (executing if the shake-ready variable is false), this embodiment checks if the shake-ready condition is met. This is conditional on the interpreted gyroscopic value's positive or negative sign realigning with the swing type; therefore, this check returns true if the interpreted gyroscopic value (from step 2820) is positive during an up-swing or primary positive swing, or negative during a down-swing or primary negative swing. If the return is true, step 2981 executes next; otherwise step 2981 may be bypassed this reading.

In step 2981 (executing if the shake-ready variable is false, but the shake-ready condition is met), this embodiment sets the shake-ready variable true. This leaves the system ready to count another shake if one is later detected in another reading within the current swing.

### Each Reading—Updating Mid-Swing Values

As this embodiment proceeds through FIG. 23's logic (executed each reading), step 2332 (conditional upon the sensor assembly's status being “moving”) updates a variety of mid-swing values which may be checked for various steps of the system's motion analysis, and may also be used for predicting changes in cadence before half-swing or full swing completions. The logic for this is expanded in FIG. 30 and described in detail here.

In step 3010, this embodiment updates the timer for the current full swing's duration or half-swing duration (depending on whether or not half-swings are being tracked, as determined in step 2430).

In step 3020, this embodiment updates the current relative angle. This entails taking the interpreted gyroscopic value (from step 2820) and adding it to the relative angle value as it was set in the previous reading.

In step 3030, this embodiment updates the current swing's range-of-motion. This step compares the relative angle from step 3020 to cached values for the highest and lowest relative angles observed in the current swing; if the current relative angle exceeds either, then the exceeded value is overwritten with the current relative angle. The range-of-motion is then calculated as the difference between the highest and lowest relative angles observed in the current swing. Additionally, this step caches the current swing's rotation rate (in degrees-per-second) as its range-of-motion divided by its duration.

In step 3040, this embodiment checks if the reading's total acceleration (the sum of the absolute values of the measurements on each accelerometer axis) is a new high in the current swing, and if so, the reading's total acceleration is cached as a new high for the current swing.

In step 3050, this embodiment checks if a half-swing response is called for. This is conditional upon half-swings currently being checked (as determined in step 2430), and upon the current swing's range-of-motion exceeding the half-swing expected range-of-motion (as set in step 2420) for the first time in the current swing. If that condition is met, this embodiment increases the repetition count by 0.25, calculates a new cadence value as 30 divided by the half-swing duration, and resets the half-swing timer.

In decision 3060, this embodiment checks if the roll is unknown. This is a simple variable check-in this embodiment, the roll is cached as an enumerator whose possible values include “Awaiting Motion” and “Analyzing” (used if the pitch is upright), either of which results in a return of true here. If the return is true, then step 3061 executes next; otherwise, otherwise, step 3080 executes next.

In step 3061 (executing if the sensor assembly's roll is unknown), this embodiment updates mid-swing values for use in upright roll estimation (as described in steps 2611-2640). This entails adding the current reading's raw gyroscopic values for the perpendicular and transverse axes to their respective cached sums for the current swing.

In decision 3070, this embodiment checks if the reading's vertical acceleration is the new highest value in the current swing. (For this embodiment, as mentioned in step 2220's description, “vertical acceleration” is measured by accelerometer values on the proximal axis. To reduce oversensitivity to slight bumps, adjustments of user posture, etc., this embodiment calculates vertical acceleration as an average of accelerometer values on the proximal axis from the last 0.1 second, though alternative embodiments may handle the value differently.) If the return is true, step 3071 executes next; otherwise, step 3080 executes next.

In step 3071 (executing if the reading's vertical acceleration exceeds the previous high for the current swing), this embodiment caches the reading's vertical acceleration value as the new high in the current swing. (This may be used for swing direction estimation if the sensor assembly's pitch is upright, as described for step 2461.)

In step 3080, this embodiment uses some of the values set in steps 3010 and 3030 to check if a change in cadence is predicted. A decrease in cadence may be predicted if the current full swing or half-swing duration value exceeds, respectively, the full swing or half-swing duration value that correlates with the current cadence value. An increase in cadence may be predicted if the current swing's rotation rate exceeds a threshold relative to the rotation rates of recently completed swings. Specifically, this embodiment divides the current swing's rotation rate by the greater of either the average rotation rate of the last four swings or the highest rotation rate monitored in the last completed swing, with a cadence increase predicted any time the quotient exceeds 1.0, and with the intensity of the increase predicted by the extent to which the quotient exceeds 1.0 (though alternative embodiments may use other thresholds).

Collectively, the mid-swing values set and analyzed in steps 3010-3080 assist with multiple areas of the embodiment's functionality, facilitating various motion detections and the calculations of activity statistics that may be used by developers to trigger a variety of software responses.

What is claimed is:

1. A method for using any repetitive athletic activity as input to a software routine running on a processor, the activity's motions sensed by a motion sensor assembly interfaced with the processor, the sensor assembly's hardware comprising one or more components of a group consisting of (i) a gyroscope component and (ii) an accelerometer component, each component providing readings with values on three local Cartesian axes, the sensor assembly removably affixed on any limb segment moved by the activity in repeated swings, with operability adaptable to any sensor assembly attachment position and orientation relative to the limb segment within the intentions of the software routine's developer, the method comprising:
gathering calibration data by at least one calibration method chosen from the group consisting of (i) a first calibration method, wherein the sensor assembly's axes are designated proximal, perpendicular, and transverse, that ascertains aspects of the sensor assembly's orientation and determines any one or more results of a group consisting of primary, secondary, and tertiary axis identifications, and sign multipliers for the proximal, perpendicular, and transverse axes (with “sign multipliers” referring to signifiers of whether values on an axis should be multiplied by −1 when determining direction of motion), (ii) a second calibration method, operable with the sensor assembly at any orientation relative to the limb segment, that samples motion data on multiple axes to determine any one or more results of a group consisting of a hierarchy of primary, secondary, and tertiary axis identifications, and primary-secondary, primary-tertiary, and secondary-tertiary sign multipliers, and (iii) a third calibration method, operable with the sensor assembly at any orientation relative to the limb segment, that determines, on an axis-by-axis basis, whether to monitor measurements on an axis when tracking the intended activity;
detecting progressions from one swing to another via any one or more detection methods chosen from a group consisting of (i) detecting changes of direction in the sensor assembly's motion, with each direction change indicating the completion of one swing and the start of another, with direction changes detected via any one or more direction-change detection methods chosen from a group consisting of (a) detecting when signs change from positive to negative or vice versa for an interpreted gyroscopic value calculated each reading, with each reading's interpreted gyroscopic value derived from multiple axes via one of a first gyroscopic interpretation method (based on sensor assembly orientation) and a second gyroscopic interpretation method (based on a hierarchy of the axes), with the interpreted value's absolute value indicating degrees rotated and the positive or negative sign indicating direction rotated, (b) detecting when gyroscopic values on any individual axis change in sign from positive to negative or vice versa, (c) detecting when gyroscopic values on any axis transition from decreasing in absolute value to increasing in absolute value from one reading to another, (d) detecting when accelerometer value deltas between one reading and another on an axis change in sign from positive to negative, and (e) detecting when accelerometer values on an axis transition from getting further from their ongoing average value to getting closer to their ongoing average value, and (ii) detecting the mid-point of each swing as the point when motion on an axis measuring the swing has peaked in speed during the swing, with each mid-point detection indicating the activity has progressed from one swing to another since the previous mid-point detection, with mid-points detected via any one or more peak-speed detection methods chosen from a group consisting of (a) detecting when gyroscopic values on any axis transition from increasing in absolute value to decreasing in absolute value from one reading to another, (b) detecting when accelerometer value deltas between one reading and another on an axis transition from decreasing in absolute value to increasing in absolute value, and (c) detecting when accelerometer values on an axis transition from getting closer to their ongoing average value to getting further from their ongoing average value;
calculating any one or more statistics of a group consisting of (i) repetition count, calculated as total completed swings divided by the number of swings per repetition, (ii) relative angle of the sensor assembly, starting as a reference value when the software initiates, the reference value being set by developer preference or based on orientation data from the first calibration method, and then calculated for each new reading as the prior relative angle plus the new reading's degrees rotated, (iii) swing range-of-motion, calculated by a calculation method chosen from the group consisting of (a) the sum of degrees rotated in the readings during a swing and (b) the difference between the highest and lowest relative angles of the sensor assembly during a swing, (iv) swing rotation rate, calculated as a swing's range-of-motion divided by its duration, (v) recent swing ranges, cached as the range-of-motion values of at least the last two completed swings, (vi) expected range-of-motion, calculated as an average of recent swing ranges, (vii) half-swing duration, calculated by a calculation method chosen from the group consisting of (a) the time taken for a swing's mid-point to be detected, or (b) the time taken for a current swing range-of-motion value to exceed half the expected range-of-motion value, (viii) swing duration, calculated by a calculation method chosen from the group consisting of (a) the time elapsed between a swing's start and completion, or (b) the time elapsed between two consecutive detections of swing mid-points, (ix) repetition duration, calculated by a calculation method chosen from the group consisting of (a) determining time elapsed while performing the number of swings per repetition, (b) calculating a single swing's duration multiplied by the number of swings per repetition, and (c) calculating twice a half-swing's duration multiplied by the number of swings per repetition, and (x) cadence, calculated by a calculation method chosen from the group consisting of (a) a unit of time numerator divided by repetition duration, (b) a unit of time numerator divided by the number of swings per repetition, then further divided by swing duration, and (c) half a unit of time numerator divided by the number of swings per repetition, then further divided by half-swing duration; and
outputting the calculated statistics as input to the software routine.

2. The method of claim 1, wherein the first calibration method designates the sensor assembly's axes as proximal, transverse, and perpendicular, the proximal axis running primarily along the limb segment's proximal-to-distal axial dimension, the perpendicular axis passing perpendicularly through the limb segment, and the transverse axis being orthogonal to the other two axes with its axial line tangential to the limb segment's surface, the first calibration method then ascertaining any one or more aspects of sensor assembly orientation from the group consisting of (i) the proximal axis local positive direction of gyroscopic rotation, (ii) the proximal axis local positive direction of linear acceleration, (iii) the perpendicular axis local positive direction of gyroscopic rotation, (iv) the perpendicular axis local positive direction of linear acceleration, (v) the transverse axis local positive direction of gyroscopic rotation, (vi) the transverse axis local positive direction of linear acceleration, (vii) the sensor assembly's pitch, and (viii) the sensor assembly's roll about the proximal axis, the aspects of sensor assembly orientation facilitating determination of any one or more results chosen from a group consisting of (i) a primary axis identification, selected from the proximal, perpendicular, and transverse axes, being the axis expected to detect the most motion during performance of the intended activity, (ii) a secondary axis identification, selected from the proximal, perpendicular, and transverse axes, being the axis expected to detect the second most motion during performance of the intended activity, (iii) a tertiary axis identification, selected from the proximal, perpendicular, and transverse axes, being the axis expected to detect the second most motion during performance of the intended activity, (iv) a proximal axis sign multiplier that signifies, based on the orientation relative to the user of local positive rotation on the proximal axis, whether gyroscopic rotation on the proximal axis should be multiplied by −1 for a positive result to indicate rotation in a known direction relative to the user and a negative result to indicate rotation in the opposite direction relative to the user, (v) a perpendicular axis sign multiplier that signifies, based on the orientation relative to the user of local positive rotation on the perpendicular axis given the sensor assembly's roll, whether gyroscopic rotation on the perpendicular axis should be multiplied by −1 for a positive result to indicate rotation in a known direction relative to the user and a negative result to indicate rotation in the opposite direction relative to the user, (vi) a transverse axis sign multiplier that signifies, based on the orientation relative to the user of local positive rotation on the transverse axis given the sensor assembly's roll, whether gyroscopic rotation on the transverse axis should be multiplied by −1 for a positive result to indicate rotation in a known direction relative to the user and a negative result to indicate rotation in the opposite direction relative to the user, the first calibration method ascertaining aspects of sensor assembly orientation via any one or more ascertainment methods chosen from the group consisting of:
(i) assuming use of developer-intended instructions for any aspects of the sensor assembly's orientation,
(ii) estimating the extent to which the sensor assembly's pitch is upright via accelerometer response to the pull of gravity, with stronger gravitational pull on the proximal axis indicating a pitch that is more upright, and stronger gravitational pull on the non-proximal axes indicating a pitch that is less upright, the estimation being expressed as one of a group consisting of (a) a numeric value indicating how upright the pitch is, and (b) a toggleable value indicating whether the pitch is upright or angled (the pitch being regarded as “angled” in the case that it is other than upright), as determined by estimating the pitch in the context of an upright/angled threshold;
(iii) estimating (in the case that local positive directions of linear acceleration are known for the perpendicular and transverse axes, and the sensor assembly's pitch is sufficiently angled for gravity to pull in a consistent direction on the perpendicular axis, transverse axis, or both perpendicular and transverse axes) the sensor assembly's roll about the proximal axis based on gravity's pull on the perpendicular and transverse axes relative to their local positive directions of linear acceleration;
(iv) estimating (in the case that local positive directions of gyroscopic rotation are known for the perpendicular and transverse axes, the sensor assembly's pitch is upright to the point that gravity does not pull in a consistent direction on the perpendicular or transverse axis, and the intended activity moves the sensor assembly in forward swings with a high mid-point and backward swings with a low mid-point) the sensor assembly's roll about the proximal axis by identifying swing directions as forward or backward, these directions being indicated by which of the last two completed swings more strongly indicated a downward-to-upward direction change, such a change being signified by acceleration on the proximal axis against the pull of gravity, with the swing that more strongly indicated a downward-to-upward direction change being identified as backward and vice versa, evaluating whether the sensor assembly's roll is orthogonal or diagonal (with “orthogonal” referring to regions of orientation the developer has deemed primarily front, back, left, or right, and “diagonal” referring to regions of orientation the developer has deemed mixes of front-or-back and left-or-right) by checking the extent to which rotation on one non-proximal axis exceeds rotation on the other non-proximal axis, with a result of “orthogonal” if the extent is over a threshold and “diagonal” if the extent is under a threshold, the threshold indicating a greater axis rotation over at least the full value of the lesser axis rotation (with thresholds that only slightly exceed a one-to-one match favoring the result of “diagonal” and higher thresholds making the result of “diagonal” increasingly selective),
evaluating whether the sensor assembly's roll is front or back by analyzing the positive or negative signs of transverse axis gyroscopic values during forward and backward swings (the known local positive direction meaning positive values during a forward swing would indicate one orientation, and negative values during a forward swing would indicate the other), evaluating whether the sensor assembly's roll is left or right by analyzing the positive or negative signs of perpendicular axis gyroscopic values during forward and backward swings (the known local positive direction meaning positive values during a forward swing would indicate one orientation, and negative values during a forward swing would indicate the other), ultimately estimating the sensor assembly's roll through the combined orthogonal-or-diagonal, front-or-back, and left-or-right evaluations; and
(v) requesting user-reported indicators for any aspects of the sensor assembly's orientation.

3. The method of claim 1, wherein the second calibration method samples readings on the sensor hardware's axes to establish and analyze an axis hierarchy, using any one or more analysis methods from a group consisting of:
(i) identifying the primary axis as the axis with the greatest sampled gyroscopic rotation for monitoring the activity via gyroscopic values;
(ii) identifying the secondary axis as the axis with the second greatest sampled gyroscopic rotation;
(iii) identifying the tertiary axis as the axis with the least sampled gyroscopic rotation;
(iv) identifying the primary axis as the axis with the greatest sampled linear acceleration;
(v) identifying the secondary axis as the axis with the second greatest linear acceleration;
(vi) identifying the tertiary axis as the axis with the least sampled linear acceleration;
(vii) setting the primary-secondary sign multiplier to signify whether one of the primary and secondary axes should have its values multiplied by −1 such that resulting values on the primary and secondary axes match in sign during performance of the intended activity, with the determination being to multiply by −1 if the positive and negative signs of values sampled on the primary and secondary axes differ more often than they match;
(viii) setting the primary-tertiary sign multiplier to signify whether one of the primary and tertiary axes should have its values multiplied by −1 such that resulting values on the primary and tertiary axes match in sign during performance of the intended activity, with the determination being to multiply by −1 if the positive and negative signs of values sampled on the primary and tertiary axes differ more often than they match;
(ix) setting the secondary-tertiary sign multiplier to signify whether one of the secondary and tertiary axes should have its values multiplied by −1 such that resulting values on the secondary and tertiary axes match in sign during performance of the intended activity, with the determination being to multiply by −1 if the positive and negative signs of values sampled on the secondary and tertiary axes differ more often than they match.

4. The method of claim 1, wherein the third calibration method determines, on an axis-by-axis basis, whether to monitor measurements on an axis when tracking the intended activity, via any one or more determination methods chosen from a group consisting of:
(i) determining whether to monitor an axis based on whether it meets a threshold of gyroscopic rotation;
(ii) determining whether to monitor an axis based on whether it meets a threshold of linear acceleration;
(iii) determining whether to monitor an axis based on its likelihood to detect motion in the performance of the intended activity, as indicated by the first calibration method's aspects of orientation;
(iv) determining whether to monitor an axis based on its likelihood to detect motion in the performance of the intended activity, as indicated by the second calibration method's axis hierarchy;
(v) determining whether to monitor an axis based on developer preference; and
(i) determining whether to monitor an axis based on user input.

5. The method of claim 1, wherein the first gyroscopic interpretation method sets the interpreted gyroscopic value as a sum of interpreted gyroscopic values from multiple axes, each axis value being “interpreted” in the sense of being potentially multiplied by −1 depending on the sensor assembly's orientation (as established by the first calibration method, with the proximal axis running primarily along the limb segment's proximal-to-distal axial dimension, the perpendicular axis passing perpendicularly through the limb segment, the transverse axis being orthogonal to the other two axes with its axial line tangential to the limb segment's surface, and with sign multipliers set for the axes), the first gyroscopic interpretation method comprising:
adding together any combination of (i) the gyroscopic value of the proximal axis, potentially multiplied by −1 if so indicated by the proximal axis sign multiplier, (ii) the gyroscopic value of the perpendicular axis, potentially multiplied by −1 if so indicated by the perpendicular axis sign multiplier, and (iii) the gyroscopic value of the transverse axis, potentially multiplied by −1 if so indicated by the transverse axis sign multiplier, to calculate an interpreted sum wherein a positive value measures degrees swung in a known direction relative to the user and a negative value measures degrees swung in the opposite direction relative to the user.

6. The method of claim 1, wherein the second gyroscopic interpretation method sets the interpreted gyroscopic value as a sum of interpreted gyroscopic values from multiple axes, each axis value being “interpreted” in the sense of being potentially multiplied by −1 depending on sample results from the second calibration method, the second gyroscopic interpretation method using any calculation method chosen from a group consisting of:
(i) adding together any combination of (a) the direct gyroscopic value for the primary axis, (b) the gyroscopic value for the secondary axis, potentially multiplied by −1 if so indicated by the primary-secondary sign multiplier, and (c) the gyroscopic value for the tertiary axis, potentially multiplied by −1 if so indicated by the primary-secondary sign multiplier, to calculate an interpreted sum wherein a positive value measures degrees swung in a direction causing positive gyroscopic values on the primary axis and a negative result measures degrees swung in a direction causing negative gyroscopic values on the primary axis;
(ii) adding together any combination of (a) the direct gyroscopic value for the secondary axis, (b) the gyroscopic value for the primary axis, potentially multiplied by −1 if so indicated by the primary-secondary sign multiplier, and (c) the gyroscopic value for the tertiary axis, potentially multiplied by −1 if so indicated by the secondary-tertiary sign multiplier to calculate an interpreted sum wherein a positive value measures degrees swung in a direction causing positive gyroscopic values on the secondary axis and a negative result measures degrees swung in a direction causing negative gyroscopic values on the secondary axis; and
(iii) adding together any combination of (a) the direct gyroscopic value for the tertiary axis, (b) the gyroscopic value for the primary axis, potentially multiplied by −1 if so indicated by the primary-tertiary sign multiplier, and (c) the gyroscopic value for the secondary axis, potentially multiplied by −1 if so indicated by the secondary-tertiary sign multiplier to calculate an interpreted sum value wherein a positive value measures degrees swung in a direction causing positive gyroscopic values on the tertiary axis and a negative result measures degrees swung in a direction causing negative gyroscopic values on the tertiary axis.

7. The method of claim 1, further comprising a detection method for identifying motion lateral to the intended activity, via a data comparison chosen depending on whether overall gyroscopic rotation during the intended activity is expected to be single-axis or multi-axis (the “single-axis” or “multi-axis” status being determined by one of a group of evaluation methods described below, with the rotation being regarded as “single-axis” in the case that gyroscopic readings from the intended activity are primarily concentrated on one axis, and “multi-axis” in the case that gyroscopic readings from the intended activity are relatively balanced on at least two axes), the detection method comprising:
determining whether the gyroscopic rotation from the intended activity is expected to be single-axis or multi-axis by an evaluation method chosen from the group consisting of (i) checking whether past gyroscopic rotation on the primary axis exceeded a threshold greater than past gyroscopic rotation on the secondary axis (with thresholds that only slightly exceed a one-to-one match favoring the result of “single-axis” and higher thresholds making the result of “single-axis” increasingly selective) and (ii) in the case the sensor assembly has a designated proximal axis aligned with the limb segment's proximal-to-distal axial dimension, analyzing accelerometer readings on the two non-proximal axes, with the result of “single-axis” indicated by the absolute value of accelerometer readings on one non-proximal axis exceeding a threshold greater than the absolute value of accelerometer readings on the other non-proximal axis (with thresholds that only slightly exceed a one-to-one match favoring the result of “single-axis” and higher thresholds making the result of “single-axis” increasingly selective);
comparing (in the case that rotation from the intended activity is expected to be single-axis) levels of current gyroscopic rotation on the primary and secondary axes, with lateral motion detected if current secondary axis rotation exceeds a threshold relative to current primary axis rotation (with higher thresholds resulting in more selective detection of lateral motion); and
comparing (in the case that rotation from the intended activity is expected to be multi-axis, and the first or second gyroscopic interpretation methods are used to calculate the interpreted gyroscopic value) the positive or negative signs of the addend values added together when calculating the interpreted gyroscopic value, with lateral motion detected if the signs of the addend values do not match.

8. The method of claim 1, further comprising a detection method for identifying shakes as distinct from swing completions, the detection method comprising at least one identification method chosen from a group consisting of:
(i) identifying individual, mid-swing shakes by establishing a minimum range-of-motion requirement less than the greatest of the recent swing ranges (with lower requirements resulting in a smaller phase in which shakes can be detected), then checking, when an interpreted gyroscopic sum indicates a change of rotational direction, whether the current swing's range-of-motion is under the minimum range-of-motion requirement, with a change of rotational direction identified as a shake rather than a swing completion if the minimum range-of-motion requirement has not been met; and
(ii) identifying repetitive shaking motion by reviewing recent swing ranges to see if a short-swing count threshold has been met regarding how many recent swing ranges have been under a short-swing range-of-motion threshold, the short-swing count threshold being at least 2 recent swings and the short-swing range-of-motion threshold being less than the expected range-of-motion (with higher short-swing count thresholds and lower short-swing range-of-motion thresholds making detection more selective), with recently completed swings identified as repetitive shaking motion rather than performance of the intended activity if the short-swing count threshold has been met.

9. The method of claim 1, further comprising a detection method for identifying sensor assembly disruptions (the term “disruption” referring to a change in the sensor assembly's attachment status that interferes with monitoring the intended activity) such that sensor assembly disruptions are not mistaken for performance of the intended activity and may trigger software response, the detection method comprising at least one identification method chosen from a group consisting of:
identifying disruptions by a dual-value check that evaluates whether any current or recent swing's range-of-motion and a highest total acceleration (the term “total acceleration” meaning the sum of the absolute values of the measurements on each accelerometer axis in a reading) both exceed respective, moderate thresholds, the range-of-motion moderate threshold being greater than the last completed swing's range-of-motion and the total acceleration moderate threshold being greater than the last completed swing's highest monitored total acceleration value (with higher values for both moderate thresholds making detection more selective), with a sensor assembly disruption detected any time a swing's range-of-motion and highest total acceleration both exceed their respective, moderate thresholds; and
identifying disruptions by a single-value check that evaluates whether a reading's total acceleration exceeds a severe threshold (greater than the moderate total acceleration threshold if the dual-value check is also in use, and with higher severe thresholds making detection more selective), with a sensor assembly disruption detected any time a reading's total acceleration exceeds the severe threshold.

10. The method of claim 1, further comprising a prediction method of anticipating changes in cadence before a new repetition duration, swing duration, or half-swing duration value is calculated, the prediction method comprising:
predicting a decrease in cadence by a monitoring method chosen from the group consisting of (i) monitoring for an in-progress half-swing duration value exceeding the half-swing duration value that correlates with the most recently calculated cadence value, (ii) monitoring for an in-progress swing duration value exceeding the swing duration value that correlates with the most recently calculated cadence value, and (iii) monitoring for an in-progress repetition duration value exceeding the repetition duration value that correlates with the most recently calculated cadence value; and
predicting an increase in cadence by monitoring swing rotation rates, with a cadence increase predicted any time an in-progress swing's rotation rate exceeds a threshold greater than the lowest rotation rate of recently completed swings (with higher thresholds making prediction more selective, and with the intensity of the increase predicted by the extent to which the in-progress rotation rate exceeds the threshold).

11. A non-transitory, computer-readable storage medium having stored thereon a software program which, when executed by a processor, is operable for performing a method for using any repetitive athletic activity as input to a software routine running on a processor, the activity's motions sensed by a motion sensor assembly interfaced with the processor, the sensor assembly's hardware comprising one or more components of a group consisting of (i) a gyroscope component and (ii) an accelerometer component, each component providing readings with values on three local Cartesian axes, the sensor assembly removably affixed on any limb segment moved by the activity in repeated swings, with operability adaptable to any sensor assembly attachment position and orientation relative to the limb segment within the intentions of the software routine's developer, the method comprising:
gathering calibration data by at least one calibration method chosen from the group consisting of (i) a first calibration method, wherein the sensor assembly's axes are designated proximal, perpendicular, and transverse, that ascertains aspects of the sensor assembly's orientation and determines any one or more results of a group consisting of primary, secondary, and tertiary axis identifications, and sign multipliers for the proximal, perpendicular, and transverse axes (with “sign multipliers” referring to signifiers of whether values on an axis should be multiplied by −1 when determining direction of motion), (ii) a second calibration method, operable with the sensor assembly at any orientation relative to the limb segment, that samples motion data on multiple axes to determine any one or more results of a group consisting of a hierarchy of primary, secondary, and tertiary axis identifications, and primary-secondary, primary-tertiary, and secondary-tertiary sign multipliers, and (iii) a third calibration method, operable with the sensor assembly at any orientation relative to the limb segment, that determines, on an axis-by-axis basis, whether to monitor measurements on an axis when tracking the intended activity;
detecting progressions from one swing to another via any one or more detection methods chosen from a group consisting of (i) detecting changes of direction in the sensor assembly's motion, with each direction change indicating the completion of one swing and the start of another, with direction changes detected via any one or more direction-change detection methods chosen from a group consisting of (a) detecting when signs change from positive to negative or vice versa for an interpreted gyroscopic value calculated each reading, with each reading's interpreted gyroscopic value derived from multiple axes via one of a first gyroscopic interpretation method (based on sensor assembly orientation) and a second gyroscopic interpretation method (based on a hierarchy of the axes), with the interpreted value's absolute value indicating degrees rotated and the positive or negative sign indicating direction rotated, (b) detecting when gyroscopic values on any individual axis change in sign from positive to negative or vice versa, (c) detecting when gyroscopic values on any axis transition from decreasing in absolute value to increasing in absolute value from one reading to another, (d) detecting when accelerometer value deltas between one reading and another on an axis change in sign from positive to negative, and (e) detecting when accelerometer values on an axis transition from getting further from their ongoing average value to getting closer to their ongoing average value, and (ii) detecting the mid-point of each swing as the point when motion on an axis measuring the swing has peaked in speed during the swing, with each mid-point detection indicating the activity has progressed from one swing to another since the previous mid-point detection, with mid-points detected via any one or more peak-speed detection methods chosen from a group consisting of (a) detecting when gyroscopic values on any axis transition from increasing in absolute value to decreasing in absolute value from one reading to another, (b) detecting when accelerometer value deltas between one reading and another on an axis transition from decreasing in absolute value to increasing in absolute value, and (c) detecting when accelerometer values on an axis transition from getting closer to their ongoing average value to getting further from their ongoing average value;
calculating any one or more statistics of a group consisting of (i) repetition count, calculated as total completed swings divided by the number of swings per repetition, (ii) relative angle of the sensor assembly, starting as a reference value when the software initiates, the reference value being set by developer preference or based on orientation data from the first calibration method, and then calculated for each new reading as the prior relative angle plus the new reading's degrees rotated, (iii) swing range-of-motion, calculated by a calculation method chosen from the group consisting of (a) the sum of degrees rotated in the readings during a swing and (b) the difference between the highest and lowest relative angles of the sensor assembly during a swing, (iv) swing rotation rate, calculated as a swing's range-of-motion divided by its duration, (v) recent swing ranges, cached as the range-of-motion values of at least the last two completed swings, (vi) expected range-of-motion, calculated as an average of recent swing ranges, (vii) half-swing duration, calculated by a calculation method chosen from the group consisting of (a) the time taken for a swing's mid-point to be detected, or (b) the time taken for a current swing range-of-motion value to exceed half the expected range-of-motion value, (viii) swing duration, calculated by a calculation method chosen from the group consisting of (a) the time elapsed between a swing's start and completion, or (b) the time elapsed between two consecutive detections of swing mid-points, (ix) repetition duration, calculated by a calculation method chosen from the group consisting of (a) determining time elapsed while performing the number of swings per repetition, (b) calculating a single swing's duration multiplied by the number of swings per repetition, and (c) calculating twice a half-swing's duration multiplied by the number of swings per repetition, and (x) cadence, calculated by a calculation method chosen from the group consisting of (a) a unit of time numerator divided by repetition duration, (b) a unit of time numerator divided by the number of swings per repetition, then further divided by swing duration, and (c) half a unit of time numerator divided by the number of swings per repetition, then further divided by half-swing duration; and
outputting the calculated statistics as input to the software routine.

12. The storage medium of claim 11, wherein the operability of the software for the first calibration method designates the sensor assembly's axes as proximal, transverse, and perpendicular, the proximal axis running primarily along the limb segment's proximal-to-distal axial dimension, the perpendicular axis passing perpendicularly through the limb segment, and the transverse axis being orthogonal to the other two axes with its axial line tangential to the limb segment's surface, the first calibration method then ascertaining any one or more aspects of sensor assembly orientation from the group consisting of (i) the proximal axis local positive direction of gyroscopic rotation, (ii) the proximal axis local positive direction of linear acceleration, (iii) the perpendicular axis local positive direction of gyroscopic rotation, (iv) the perpendicular axis local positive direction of linear acceleration, (v) the transverse axis local positive direction of gyroscopic rotation, (vi) the transverse axis local positive direction of linear acceleration, (vii) the sensor assembly's pitch, and (viii) the sensor assembly's roll about the proximal axis, the aspects of sensor assembly orientation facilitating determination of any one or more results chosen from a group consisting of (i) a primary axis identification, selected from the proximal, perpendicular, and transverse axes, being the axis expected to detect the most motion during performance of the intended activity, (ii) a secondary axis identification, selected from the proximal, perpendicular, and transverse axes, being the axis expected to detect the second most motion during performance of the intended activity, (iii) a tertiary axis identification, selected from the proximal, perpendicular, and transverse axes, being the axis expected to detect the second most motion during performance of the intended activity, (iv) a proximal axis sign multiplier that signifies, based on the orientation relative to the user of local positive rotation on the proximal axis, whether gyroscopic rotation on the proximal axis should be multiplied by −1 for a positive result to indicate rotation in a known direction relative to the user and a negative result to indicate rotation in the opposite direction relative to the user, (v) a perpendicular axis sign multiplier that signifies, based on the orientation relative to the user of local positive rotation on the perpendicular axis given the sensor assembly's roll, whether gyroscopic rotation on the perpendicular axis should be multiplied by −1 for a positive result to indicate rotation in a known direction relative to the user and a negative result to indicate rotation in the opposite direction relative to the user, (vi) a transverse axis sign multiplier that signifies, based on the orientation relative to the user of local positive rotation on the transverse axis given the sensor assembly's roll, whether gyroscopic rotation on the transverse axis should be multiplied by −1 for a positive result to indicate rotation in a known direction relative to the user and a negative result to indicate rotation in the opposite direction relative to the user, the first calibration method ascertaining aspects of sensor assembly orientation via any one or more ascertainment methods chosen from the group consisting of:
(i) assuming use of developer-intended instructions for any aspects of the sensor assembly's orientation,
(ii) estimating the extent to which the sensor assembly's pitch is upright via accelerometer response to the pull of gravity, with stronger gravitational pull on the proximal axis indicating a pitch that is more upright, and stronger gravitational pull on the non-proximal axes indicating a pitch that is less upright, the estimation being expressed as one of a group consisting of (a) a numeric value indicating how upright the pitch is, and (b) a toggleable value indicating whether the pitch is upright or angled (the pitch being regarded as “angled” in the case that it is other than upright), as determined by estimating the pitch in the context of an upright/angled threshold;
(iii) estimating (in the case that local positive directions of linear acceleration are known for the perpendicular and transverse axes, and the sensor assembly's pitch is sufficiently angled for gravity to pull in a consistent direction on the perpendicular axis, transverse axis, or both perpendicular and transverse axes) the sensor assembly's roll about the proximal axis based on gravity's pull on the perpendicular and transverse axes relative to their local positive directions of linear acceleration;
(iv) estimating (in the case that local positive directions of gyroscopic rotation are known for the perpendicular and transverse axes, the sensor assembly's pitch is upright to the point that gravity does not pull in a consistent direction on the perpendicular or transverse axis, and the intended activity moves the sensor assembly in forward swings with a high mid-point and backward swings with a low mid-point) the sensor assembly's roll about the proximal axis by identifying swing directions as forward or backward, these directions being indicated by which of the last two completed swings more strongly indicated a downward-to-upward direction change, such a change being signified by acceleration on the proximal axis against the pull of gravity, with the swing that more strongly indicated a downward-to-upward direction change being identified as backward and vice versa, evaluating whether the sensor assembly's roll is orthogonal or diagonal (with “orthogonal” referring to regions of orientation the developer has deemed primarily front, back, left, or right, and “diagonal” referring to regions of orientation the developer has deemed mixes of front-or-back and left-or-right) by checking the extent to which rotation on one non-proximal axis exceeds rotation on the other non-proximal axis, with a result of “orthogonal” if the extent is over a threshold and “diagonal” if the extent is under a threshold, the threshold indicating a greater axis rotation over at least the full value of the lesser axis rotation (with thresholds that only slightly exceed a one-to-one match favoring the result of “diagonal” and higher thresholds making the result of “diagonal” increasingly selective),
evaluating whether the sensor assembly's roll is front or back by analyzing the positive or negative signs of transverse axis gyroscopic values during forward and backward swings (the known local positive direction meaning positive values during a forward swing would indicate one orientation, and negative values during a forward swing would indicate the other), evaluating whether the sensor assembly's roll is left or right by analyzing the positive or negative signs of perpendicular axis gyroscopic values during forward and backward swings (the known local positive direction meaning positive values during a forward swing would indicate one orientation, and negative values during a forward swing would indicate the other), ultimately estimating the sensor assembly's roll through the combined orthogonal-or-diagonal, front-or-back, and left-or-right evaluations; and
(v) requesting user-reported indicators for any aspects of the sensor assembly's orientation.

13. The storage medium of claim 11, wherein the operability of the software for the second calibration method samples readings on the sensor hardware's axes to establish and analyze an axis hierarchy, using any one or more analysis methods from a group consisting of:
(i) identifying the primary axis as the axis with the greatest sampled gyroscopic rotation for monitoring the activity via gyroscopic values;
(ii) identifying the secondary axis as the axis with the second greatest sampled gyroscopic rotation;
(iii) identifying the tertiary axis as the axis with the least sampled gyroscopic rotation;
(iv) identifying the primary axis as the axis with the greatest sampled linear acceleration;
(v) identifying the secondary axis as the axis with the second greatest linear acceleration;
(vi) identifying the tertiary axis as the axis with the least sampled linear acceleration;
(vii) setting the primary-secondary sign multiplier to signify whether one of the primary and secondary axes should have its values multiplied by −1 such that resulting values on the primary and secondary axes match in sign during performance of the intended activity, with the determination being to multiply by −1 if the positive and negative signs of values sampled on the primary and secondary axes differ more often than they match;
(viii) setting the primary-tertiary sign multiplier to signify whether one of the primary and tertiary axes should have its values multiplied by −1 such that resulting values on the primary and tertiary axes match in sign during performance of the intended activity, with the determination being to multiply by −1 if the positive and negative signs of values sampled on the primary and tertiary axes differ more often than they match;
(ix) setting the secondary-tertiary sign multiplier to signify whether one of the secondary and tertiary axes should have its values multiplied by −1 such that resulting values on the secondary and tertiary axes match in sign during performance of the intended activity, with the determination being to multiply by −1 if the positive and negative signs of values sampled on the secondary and tertiary axes differ more often than they match.

14. The storage medium of claim 11, wherein the operability of the software for the third calibration method determines, on an axis-by-axis basis, whether to monitor measurements on an axis when tracking the intended activity, via any one or more determination methods chosen from a group consisting of:
(i) determining whether to monitor an axis based on whether it meets a threshold of gyroscopic rotation;
(ii) determining whether to monitor an axis based on whether it meets a threshold of linear acceleration;
(iii) determining whether to monitor an axis based on its likelihood to detect motion in the performance of the intended activity, as indicated by the first calibration method's aspects of orientation;
(iv) determining whether to monitor an axis based on its likelihood to detect motion in the performance of the intended activity, as indicated by the second calibration method's axis hierarchy;
(v) determining whether to monitor an axis based on developer preference; and
(i) determining whether to monitor an axis based on user input.

15. The storage medium of claim 11, wherein operability of the software for the first gyroscopic interpretation method sets the interpreted gyroscopic value as a sum of interpreted gyroscopic values from multiple axes, each axis value being “interpreted” in the sense of being potentially multiplied by −1 depending on the sensor assembly's orientation (as established by the first calibration method, with the proximal axis running primarily along the limb segment's proximal-to-distal axial dimension, the perpendicular axis passing perpendicularly through the limb segment, the transverse axis being orthogonal to the other two axes with its axial line tangential to the limb segment's surface, and with sign multipliers set for the axes), the first gyroscopic interpretation method comprising:
adding together any combination of (i) the gyroscopic value of the proximal axis, potentially multiplied by −1 if so indicated by the proximal axis sign multiplier, (ii) the gyroscopic value of the perpendicular axis, potentially multiplied by −1 if so indicated by the perpendicular axis sign multiplier, and (iii) the gyroscopic value of the transverse axis, potentially multiplied by −1 if so indicated by the transverse axis sign multiplier, to calculate an interpreted sum wherein a positive value measures degrees swung in a known direction relative to the user and a negative value measures degrees swung in the opposite direction relative to the user.

16. The storage medium of claim 11, wherein operability of the software for the second gyroscopic interpretation method sets the interpreted gyroscopic value as a sum of interpreted gyroscopic values from multiple axes, each axis value being “interpreted” in the sense of being potentially multiplied by −1 depending on sample results from the second calibration method, the second gyroscopic interpretation method using any calculation method chosen from a group consisting of:
(i) adding together any combination of (a) the direct gyroscopic value for the primary axis, (b) the gyroscopic value for the secondary axis, potentially multiplied by −1 if so indicated by the primary-secondary sign multiplier, and (c) the gyroscopic value for the tertiary axis, potentially multiplied by −1 if so indicated by the primary-secondary sign multiplier, to calculate an interpreted sum wherein a positive value measures degrees swung in a direction causing positive gyroscopic values on the primary axis and a negative result measures degrees swung in a direction causing negative gyroscopic values on the primary axis;
(ii) adding together any combination of (a) the direct gyroscopic value for the secondary axis, (b) the gyroscopic value for the primary axis, potentially multiplied by −1 if so indicated by the primary-secondary sign multiplier, and (c) the gyroscopic value for the tertiary axis, potentially multiplied by −1 if so indicated by the secondary-tertiary sign multiplier to calculate an interpreted sum wherein a positive value measures degrees swung in a direction causing positive gyroscopic values on the secondary axis and a negative result measures degrees swung in a direction causing negative gyroscopic values on the secondary axis; and
(iii) adding together any combination of (a) the direct gyroscopic value for the tertiary axis, (b) the gyroscopic value for the primary axis, potentially multiplied by −1 if so indicated by the primary-tertiary sign multiplier, and (c) the gyroscopic value for the secondary axis, potentially multiplied by −1 if so indicated by the secondary-tertiary sign multiplier to calculate an interpreted sum value wherein a positive value measures degrees swung in a direction causing positive gyroscopic values on the tertiary axis and a negative result measures degrees swung in a direction causing negative gyroscopic values on the tertiary axis.

17. The storage medium of claim 11, further comprising operability of the software for a detection method for identifying motion lateral to the intended activity, via a data comparison chosen depending on whether overall gyroscopic rotation during the intended activity is expected to be single-axis or multi-axis (the “single-axis” or “multi-axis” status being determined by one of a group of evaluation methods described below, with the rotation being regarded as “single-axis” in the case that gyroscopic readings from the intended activity are primarily concentrated on one axis, and “multi-axis” in the case that gyroscopic readings from the intended activity are relatively balanced on at least two axes), the detection method comprising:
determining whether the gyroscopic rotation from the intended activity is expected to be single-axis or multi-axis by an evaluation method chosen from the group consisting of (i) checking whether past gyroscopic rotation on the primary axis exceeded a threshold greater than past gyroscopic rotation on the secondary axis (with thresholds that only slightly exceed a one-to-one match favoring the result of “single-axis” and higher thresholds making the result of “single-axis” increasingly selective) and (ii) in the case the sensor assembly has a designated proximal axis aligned with the limb segment's proximal-to-distal axial dimension, analyzing accelerometer readings on the two non-proximal axes, with the result of “single-axis” indicated by the absolute value of accelerometer readings on one non-proximal axis exceeding a threshold greater than the absolute value of accelerometer readings on the other non-proximal axis (with thresholds that only slightly exceed a one-to-one match favoring the result of “single-axis” and higher thresholds making the result of “single-axis” increasingly selective);
comparing (in the case that rotation from the intended activity is expected to be single-axis) levels of current gyroscopic rotation on the primary and secondary axes, with lateral motion detected if current secondary axis rotation exceeds a threshold relative to current primary axis rotation (with higher thresholds resulting in more selective detection of lateral motion); and
comparing (in the case that rotation from the intended activity is expected to be multi-axis, and the first or second gyroscopic interpretation methods are used to calculate the interpreted gyroscopic value) the positive or negative signs of the addend values added together when calculating the interpreted gyroscopic value, with lateral motion detected if the signs of the addend values do not match.

18. The storage medium of claim 11, further comprising operability of the software for a detection method for identifying shakes as distinct from swing completions, the detection method comprising at least one identification method chosen from a group consisting of:
(i) identifying individual, mid-swing shakes by establishing a minimum range-of-motion requirement less than the greatest of the recent swing ranges (with lower requirements resulting in a smaller phase in which shakes can be detected), then checking, when an interpreted gyroscopic sum indicates a change of rotational direction, whether the current swing's range-of-motion is under the minimum range-of-motion requirement, with a change of rotational direction identified as a shake rather than a swing completion if the minimum range-of-motion requirement has not been met; and
(ii) identifying repetitive shaking motion by reviewing recent swing ranges to see if a short-swing count threshold has been met regarding how many recent swing ranges have been under a short-swing range-of-motion threshold, the short-swing count threshold being at least 2 recent swings and the short-swing range-of-motion threshold being less than the expected range-of-motion (with higher short-swing count thresholds and lower short-swing range-of-motion thresholds making detection more selective), with recently completed swings identified as repetitive shaking motion rather than performance of the intended activity if the short-swing count threshold has been met.

19. The storage medium of claim 11, wherein the sensor assembly also includes accelerometer hardware, further comprising operability of the software for a detection method for identifying sensor assembly disruptions (the term “disruption” referring to a change in the sensor assembly's attachment status that interferes with monitoring the intended activity) such that sensor assembly disruptions are not mistaken for performance of the intended activity and may trigger software response, the detection method comprising at least one identification method chosen from a group consisting of:
identifying disruptions by a dual-value check that evaluates whether any current or recent swing's range-of-motion and a highest total acceleration (the term “total acceleration” meaning the sum of the absolute values of the measurements on each accelerometer axis in a reading) both exceed respective, moderate thresholds, the range-of-motion moderate threshold being greater than the last completed swing's range-of-motion and the total acceleration moderate threshold being greater than the last completed swing's highest monitored total acceleration value (with higher values for both moderate thresholds making detection more selective), with a sensor assembly disruption detected any time a swing's range-of-motion and highest total acceleration both exceed their respective, moderate thresholds; and
identifying disruptions by a single-value check that evaluates whether a reading's total acceleration exceeds a severe threshold (greater than the moderate total acceleration threshold if the dual-value check is also in use, and with higher severe thresholds making detection more selective), with a sensor assembly disruption detected any time a reading's total acceleration exceeds the severe threshold.

20. The storage medium of claim 11, further comprising operability of the software for a prediction method of anticipating changes in cadence before a new repetition duration, swing duration, or half-swing duration value is calculated, the prediction method comprising:
predicting a decrease in cadence by a monitoring method chosen from the group consisting of (i) monitoring for an in-progress half-swing duration value exceeding the half-swing duration value that correlates with the most recently calculated cadence value, (ii) monitoring for an in-progress swing duration value exceeding the swing duration value that correlates with the most recently calculated cadence value, and (iii) monitoring for an in-progress repetition duration value exceeding the repetition duration value that correlates with the most recently calculated cadence value; and
predicting an increase in cadence by monitoring swing rotation rates, with a cadence increase predicted any time an in-progress swing's rotation rate exceeds a threshold greater than the lowest rotation rate of recently completed swings (with higher thresholds making prediction more selective, and with the intensity of the increase predicted by the extent to which the in-progress rotation rate exceeds the threshold).
